(19) United States
(12) Patent Application Publication
(10) Pub. No.: US 2025/0222129 A1
(43) Pub. Date: Jul. 10, 2025

(54) Lipid Based Nanoparticles for Targeted Gene Delivery to the Brain

(71) Applicant: National Research Council of Canada, Ottawa (CA)

(72) Inventors: Abedelnasser Abulrob, Ottawa (CA); Danica Stanimirovic, Ottawa (CA); Umar Iqbal, Ottawa (CA); Bryan Simard, Ottawa (CA); Michel Gilbert, Ottawa (CA); Yves Durocher, Montreal (CA); Warren Wakarchuk, Edmonton (CA)

(73) Assignee: National Research Council of Canada, Ottawa, ON (CA)

(21) Appl. No.: 18/849,372

(22) PCT Filed: Mar. 21, 2023
(86) PCT No.: PCT/CA2023/050366

§ 371 Date: Sep. 20, 2024

Related U.S. Application Data
(60) Provisional application No. 63/321,880, filed on Mar. 21, 2022.

(51) Int. Cl. A61K 47/69; A61K 31/7105; A61K 47/68; B82Y 5/00; C12N 15/113

(52) U.S. Cl. CPC A61K 47/6913; A61K 31/7105; A61K 47/6843; C12N 15/113; B82Y 5/00; C12N 2310/113; C12N 2310/141; C12N 2310/531

(57) ABSTRACT

The present document describes a pharmaceutical composition comprising a) a lipid nanoparticle operable to encapsulate a therapeutic agent, comprising a core and an external surface, said therapeutic agent being encapsulated within said core; said lipid nanoparticle having a size of said lipid nanoparticle of from about 30 to about 80 nm, or a pegylated lipid comprising a distearoyl-rac-glycerol (DSG)-PEG and 1,2-distearoyl-sn-glycero-3-phosphoethanolamine-N-(DSPE)-PEG-DBCO; or a combination of: a size of from about 30 to about 80 nm and a pegylated lipid comprising a DSG-PEG and DSPE-PEG-DBCO; and b) an antibody or antigen-binding fragment thereof operable to transmigrate the blood-brain barrier (BBB), wherein the antibody or antigen-binding fragment thereof comprises complementarity determining regions (CDR1, CDR2 and CDR3), operably linked to said external surface of said lipid nanoparticle.

### CROSS-REFERENCE TO RELATED APPLICATIONS

This application claims priority of U.S. Provisional Patent Application No. 63/321,880 filed on Mar. 21, 2022, the specification of which is hereby incorporated by reference in its entirety.

### BACKGROUND

### (A) Field

The subject matter disclosed generally relates to pharmaceutical compositions comprising lipid nanoparticles for targeted delivery of therapeutics to the brain, and particularly to pharmaceutical compositions comprising nanoparticle operable to encapsulate a therapeutic agent and targeted with antibody or antigen-binding fragment thereof operable to transmigrate the blood-brain barrier (BBB).

### (b) Related Prior Art

Gene therapy is attractive as a clinical treatment for cancers and genetic disorders of both congenital and acquired origins. Efficient gene delivery systems are central to the clinical treatment of genetic disorders and cancer and have attracted considerable attention in recent years. The use of recombinant viruses as gene carriers was the focus of early studies due its high transfection efficiencies and levels of protein expressions. However, these systems are critically limited because viral proteins trigger strong immune responses. Additionally, viral delivery systems are limited in scale-up procedures. As a result, numerous nonviral gene delivery systems such as cationic lipids, polymers, dendrimers, and peptides have been developed. However, nonviral gene delivery systems exhibit significantly reduced transfection efficiencies compared to viral systems due to numerous extra- and intracellular obstacles. Therefore, many researchers continue to focus on designing safe and efficient viral delivery vectors.

Although messenger RNA (mRNA), antisense oligonucleotides (ASO) and RNA interference (RNAi) has proven to have tremendous potential as a new therapeutic strategy, there remains a need to efficiently deploy therapeutic mRNA, ASO and RNAi agents to specifically targeted sites or tissues. Accordingly, delivery systems that allow for targeted delivery to specific cell types and which are non-toxic, non-immunogenic and biodegradable are needed.

Nucleic acid therapeutics, to repair, replace, or regulate genes to prevent or treat disease, is attracting a lot of attention nowadays due to its high potential. These gene therapy treatments were initially considered good candidate for rare inherited disorders (such as mutated cystic fibrosis gene or mutant Huntingtin gene). However, it is now accepted the application of gene therapy may open treatment opportunities for even more challenging and complex diseases such as Alzheimer's or Parkinson's disease.

The central issue preventing the widespread implementation of gene therapy to treat brain diseases is its successful delivery. Gene modifying macromolecules including ASO, Small interfering RNA (siRNA), Short hairpin RNA (shRNA), mRNA, plasmid DNA (pDNA) and Cas9 protein are susceptible to breakdown in biological fluids, do not accumulate at the desired sites following systemic administration because of the blood brain barrier, and they cannot access the intracellular sites of action (i.e., the cytoplasm or the nucleus). To enable the therapeutic potential of gene modifying macromolecules to treat the nervous system, it is critical to develop brain “precision targeted” nano-delivery platforms that can facilitate transmigration across the blood brain barrier, uptake into target cells such as neurons, trigger cytosolic release and support entry into the nucleus.

Therefore, there is a need for novel pharmaceutical compositions for the targeted delivery of therapeutics to the brain.

Particularly, there is a need for novel pharmaceutical compositions for the targeted delivery of therapeutics to the brain that mitigate the shortcomings existing pharmaceutical compositions.

### SUMMARY

According to an embodiment, there is provided a pharmaceutical composition comprising:
a) a lipid nanoparticle operable to encapsulate a therapeutic agent, comprising a core and an external surface, the therapeutic agent being encapsulated within the core; the lipid nanoparticle having
   a size of the lipid nanoparticle of from about 30 to about 80 nm, or
   a pegylated lipid comprising a distearoyl-rac-glycerol (DSG)-PEG, a 1,2-distearoyl-sn-glycero-3-phosphoethanolamine-N-(DSPE)-PEG-DBCO, or a combination thereof; or
   a combination of: a size of from about 30 to about 80 nm and a pegylated lipid comprising a DSG-PEG, DSPE-PEG-DBCO, or a combination thereof; and
b) an antibody or antigen-binding fragment thereof operable to transmigrate the blood-brain barrier (BBB), wherein the antibody or antigen-binding fragment thereof comprises complementarity determining regions (CDR1, CDR2 and CDR3), operably linked to the external surface of the lipid nanoparticle.

The lipid nanoparticle may be from about 40 to about 60 nm.

The lipid nanoparticle may comprise an ionizable cationic lipid, a neutral, charged, saturated or unsaturated helper phospholipid, cholesterol, and combinations thereof.

The pegylated lipid may further comprise a 1,2-dimyristoyl-rac-glycero-3-methoxy (DMG)-PEG, a 1,2-dipalmitoyl-sn-glycero-3-phosphoethanolamine-N(DPPE)-PEG; or a combination thereof.

The pegylated lipid may comprise a PEG group having a molecular weight of about 500 to about 5000 g/mol.

The PEG group may have a molecular weight of about 2000 g/mol.

The DSG-PEG may be DSG-PEG₂₀₀₀.

The DSPE-PEG may be DSPE-PEG₂₀₀₀.

The DMG-PEG or DPPE-PEG may be DMG-PEG₂₀₀₀ and DPPE-PEG₂₀₀₀, respectively.

The ionizable lipid may be (6Z,9Z,28Z,31Z)-Heptatriaconta-6,9,28,31-tetraen-19-yl 4-(dimethylamino) butanoate (DLin-MC3-DMA), 2-[2,2-bis[(9Z,12Z)-octadeca-9,12-dienyl]-1,3-dioxolan-4-yl]-N,N-dimethylethanamine (DLin-KC2-DMA), [(4-hydroxybutyl)azanediyl]di(hexane-6,1-diyl)bis(2-hexyldecanoate) (ALC-0315), Heptadecan-9-yl 8-{(2-hydroxyethyl)[6-oxo-6-(undecyloxy)hexyl]amino}octanoate (SM-102), and combinations thereof.

The helper phospholipid may be 1,2-distearoyl-sn-glycero-3-phosphocholine (DSPC), 1,2-dioleoyl-sn-glycero-3-phosphocholine (DOPC), 1,2-Dioleoyl-sn-glycero-3-phosphoethanolamine (DOPE), 1-stearoyl-2-oleoyl-sn-glycero-3-phosphocholine (SOPC) and combinations thereof.

The antibody or antigen-binding fragment thereof operable to transmigrate the BBB may further comprise an added O-glycosylation sequon glycosylated with an O-glycan having the general formula (I):

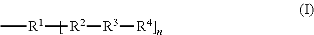

wherein
   R¹ is an initial N-acetylgalactosamine (GalNAc);
   n=1, or 2;
   R² are each independently absent, galactose (Gal), GalNAc, N-Acetylglucosamine (GlcNAc), or a sialic acid;
   R³ are each independently absent, Gal or a sialic acid; and
   R⁴ are each independently absent or a sialic acid;
wherein the O-glycan is operably linked to the external surface of the nanoparticle.

The initial GalNAc, and/or any one of the R¹, R², R³ and R⁴ may be further modified with one or more chemical group.

The chemical group may be one or more of a methyl group, an acetyl group, a sulfate group, or a combination thereof.

The sialic acid may be N-Acetylneuraminic acid (Neu5Ac), 9-azido-N-Acetylneuraminic acid (9N₃-Neu5Ac), N-azidoacetylneuraminic acid (Neu5NAz), or a combination thereof.

The n=1 and R² may be Gal.

The R³ may be a sialic acid selected from the group consisting of Neu5Ac, Neu5NAz and 9N3-Neu5Ac, and R⁴ may be absent.

The O-glycan may have the general formula (II):

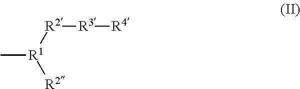

wherein
R^{2′} is Gal, or GlcNAc;
R^{3′} is Gal or a sialic acid;
R^{4′} is absent, or a sialic acid; and
R^{2″} is GlcNAc or a sialic acid.

The a R^{2′} may be Gal, a R^{3′} may be a sialic acid consisting of Neu5Ac, and a R^{4′} may be absent or a sialic acid consisting of 9N3-Neu5Ac; and a R^{2″} may be a sialic acid consisting of Neu5Ac.

The added O-glycosylation sequon may comprise an amino acid sequence comprising:
PTTDSTX₁PAPTTK, where X₁ is S or T (SEQ ID NO: 1);
FFPX₂PGP, where X₂ is S or T (SEQ ID NO: 2);
GVGVX₃ETP, where X₃ is S or T (SEQ ID NO: 3);
AAAX₄PAP, where X₄ is S or T (SEQ ID NO: 4); and
APALQPX₅QGAMPA, where X₅ is S or T (SEQ ID NO: 5), or combinations thereof.

The added O-glycosylation sequon may comprise an amino acid sequence comprising:
PTTDSTTPAPTTK (SEQ ID NO: 6), PTTDSTSPAPTTK (SEQ ID NO: 7), FFPTPGP (SEQ ID NO: 8); FFPSPGP (SEQ ID NO: 9), GVGVTETP (SEQ ID NO: 10), GVGVSETP (SEQ ID NO: 11), AAATPAP (SEQ ID NO: 12), AAASPAP (SEQ ID NO: 13); APALQPTQGAMPA (SEQ ID NO: 14), and APALQPSQGAMPA (SEQ ID NO: 15).

The added O-glycosylation sequon may be at a C-terminus of the antibody or antigen-binding fragment thereof.

The antibody or antigen-binding fragment thereof operable to transmigrate the BBB, may comprise a cysteine amino acid operable to make a thioether covalent bond, and/or an epsilon amino group operable to make an amide covalent bond, for conjugation with the nanoparticle.

The antibody or antigen-binding fragment thereof operable to transmigrate the BBB may comprise a reactive functional group for conjugation with the lipid nanoparticle.

The reactive functional group may be an azido group.

The antigen-binding fragment may be a single-domain antibody (sdAb), a fragment antigen-binding (Fab), a single-chain variable fragment (scFv), or a single-chain fragment antigen-binding (scFab).

The antibody may be an IgA, an IgD, an IgE, an IgG, or an IgM.

The antibody or antigen-binding fragment thereof may be humanized or partially humanized.

The antibody or antigen-binding fragment thereof may comprise complementarity determining regions (CDR1, CDR2 and CDR3) having the sequences:
a CDR1 sequence GFKITHYTMG (SEQ ID NO:16); CDR2 sequence RITWGGX₁X₂TX₃YSNSVKG, where X₁ is D or K, X₂ is N or D, and X₃ is F, I or L (SEQ ID NO: 17); and CDR3 sequence GSTSTAX₄PLRVDY, where X₄ is T or K (SEQ ID NO: 18).

The antibody or antigen-binding fragment thereof comprises an amino acid sequence selected from the group consisting of:

(SEQ ID NO: 19)
X₁VQLVESGGGLVQPGGSLRLSCAASGFKITHYTMGWX₂RQAP
GKX₃X₄EX₅VSRITWGGDNTFYSNSVKGRFTISRDNSKNTX₆YL
QMNSLRAEDTAVYYCAAGSTSTATPLRVDYWGQGTLVTVSS,
wherein
X₁ = D or E,
X₂ = F or V,
X₃ = E or G,
X₄ = R or L,
X₅ = F or W,
and
X₆ = L or V.

The external surface may comprise a functionalized cyclooctyne operably linking the antibody or antigen-binding fragment or the O-glycan of the antibody or antigen-binding fragment to the external surface.

The O-glycan may comprises a 9N₃-Neu5Ac moiety operably linked to the functionalized cyclooctyne.

The functionalized cyclooctyne may be dibenzocyclooctyne (DBCO), bicyclononyne (BCN), cyclooctyne (COT), monofluorinated cyclooctyne (MOFO), difluorocyclooctyne (DIFO), dimethoxyazacyclooctyne (DIMAC), dibenzoazacyclooctyne (DIBAC), biarylazacyclooctynone (BARAC), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), or combinations thereof.

The functionalized cyclooctyne may be conjugated to the pegylated lipid.

The pegylated lipid may be a pegylated phospholipid.

The pegylated phospholipid may be DSPE-PEG₂₀₀₀-X¹, wherein X¹ is the functionalized cyclooctyne.

The functionalized cyclooctyne may be DBCO, BCN, COT, or combinations thereof.

The lipid nanoparticle may comprise a molar ratio of from about 10% to about 60% of an ionizable lipid.

The lipid nanoparticle may comprise a molar ratio of from about 30% to about 50% of an ionizable lipid.

The lipid nanoparticle may comprise a molar ratio of from about 5% to about 40% of the helper lipid 1,2-distearoyl-sn-glycero-3-phosphocholine (DSPC), 1,2-Distearoyl-sn-glycero-3-phosphorylethanolamine (DSPE), 1,2-dioleoyl-sn-glycero-3-phosphocholine (DOPC), 1,2-dipalmitoyl-sn-glycero-3-phosphoethanolamine (DPPE), 1,2-Dioleoyl-sn-glycero-3-phosphoethanolamine (DOPE), 1-stearoyl-2-oleoyl-sn-glycero-3-phosphocholine (SOPC) and combinations thereof.

The lipid nanoparticle may comprise a molar ratio of the helper lipid from about 10% to about 30% of the DSPC, DSPE, DOPC, DPPE, DOPE, and SOPC, or combinations thereof.

The lipid nanoparticle may comprise a molar ratio of from about 20% to about 50% of cholesterol.

The lipid nanoparticle may comprise a molar ratio of from about 25% to 40% of cholesterol.

The lipid nanoparticle may comprise a molar ratio of from about 1% to about 10% of 1,2-dipalmitoyl-sn-glycero-3-phosphoethanolamine-N-[PEG-2000] (DPPE-PEG₂₀₀₀), distearoyl-rac-glycerol-[PEG-2000] (DSG-PEG₂₀₀₀), 1,2-dimyristoyl-rac-glycero-3-methoxy-[PEG-2000] (DMG-PEG₂₀₀₀), 1,2-distearoyl-sn-glycero-3-phosphoethanolamine-N-[PEG-2000] (DSPE-PEG₂₀₀₀).

The lipid nanoparticle may comprise a molar ratio of from about 1% to about 5% of 1,2-dipalmitoyl-sn-glycero-3-phosphoethanolamine-N-[PEG-2000] (DPPE-PEG₂₀₀₀), distearoyl-rac-glycerol-[PEG-2000] (DSG-PEG₂₀₀₀), 1,2-dimyristoyl-rac-glycero-3-methoxy-[PEG-2000] (DMG-PEG₂₀₀₀), or 1,2-distearoyl-sn-glycero-3-phosphoethanolamine-N-[PEG-2000] (DSPE-PEG₂₀₀₀).

The lipid nanoparticle may comprise a molar ratio of from about 0.05% to about 2% of DPPE-PEG₂₀₀₀-DBCO, DSG-PEG₂₀₀₀-DBCO, DMG-PEG₂₀₀₀-DBCO, DSPE-PEG₂₀₀₀-DBCO, or combinations thereof.

The lipid nanoparticle may comprise a molar ratio of from about 0.05% to about 1% of DPPE-PEG₂₀₀₀-DBCO, DSG-PEG₂₀₀₀-DBCO, DMG-PEG₂₀₀₀-DBCO, DSPE-PEG₂₀₀₀-DBCO, or combinations thereof.

The lipid nanoparticle may comprise a molar ratio of:
from about 40% to about 50% ALC-0315, DLin-MC3-DMA, ALC-0315, or a combination thereof;
from about 5% to about 10% DSPC;
from about 35% to about 40% cholesterol;
from about 1.5% to about 2.5% DPPE-PEG₂₀₀₀, DSG-PEG₂₀₀₀, DMG-PEG₂₀₀₀, or DSPE-PEG₂₀₀₀, or combinations thereof; and
from about 0.05% to about 1% of DSPE-PEG₂₀₀₀-DBCO.

The therapeutic agent may be a peptide, a polypeptide, a protein, an enzyme, an antibody, an antibody fragment, a nucleic acid, or combinations thereof.

The nucleic acid may be an antisense oligonucleotide (ASO), a duplex RNA, a single stranded RNA molecule, a ministering DNA (msDNA), a DNA plasmid, or combinations thereof.

The duplex RNA may be a small interfering RNA (siRNA), a microRNA (miRNA), or a combination thereof.

The single stranded RNA molecule may be a short hairpin RNA (shRNA), an mRNA, and anti-miRNA, or combinations thereof.

According to another embodiment, there is provided a composition comprising the pharmaceutical composition of the present invention, and a pharmaceutically acceptable diluent, carrier or excipient.

According to another embodiment, there is provided a method of delivering a therapeutic agent across the BBB, comprising administering the pharmaceutical composition according to the present invention or a composition according to the present invention to a subject in need thereof.

The therapeutic agent may be a pharmaceutical composition according to the present invention, and the method is for the treatment of a brain disease applicable for gene therapy (gene addition, silencing or editing).

According to another embodiment, there is provided a use of a pharmaceutical composition according to the present invention or a composition according to the present invention, for the delivery of a therapeutic agent in the brain of a subject in need thereof, for the treatment of a brain disease.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates single domain antibody (sdAb) click conjugation chemistry to the lipid nanoparticle (LNP), according to an embodiment of the present invention, using pre-insertion of click lipids during LNP formation, followed by antibody click reactions.

FIG. 2 illustrates single domain antibody (sdAb) click conjugation chemistry to the lipid nanoparticle (LNP), according to an embodiment of the present invention using post-insertion of antibody linked click lipids micelles into pre-formed LNPs.

FIG. 3 illustrates LNP stability/cargo release in vitro by measuring cargo stability in 50% serum at 37° C.

FIG. 4 illustrates an assay to show that intact LNPs-FC5 cross the BBB in vitro.

FIG. 5 illustrates the effect of increasing FC5 density on LNPs on the amount of encapsulated Cy5-ASO crossing the BBB model in vitro.

FIG. 6 illustrates the effect of increased density of IGF1R-sdAb (shown as IGF1R) on LNPs on the efficiency of BBB transmigration and ASO delivery in vitro after 3 h (left) and 24 h (right).

FIG. 7 illustrates the detection of FC5 in LNPs-FC5 by Western blot after passage to the bottom chamber of the in vitro rat BBB model.

FIG. 8 illustrates the transport of ASO across the in vitro BBB encapsulated in IGF1R-targeted LNPs or non-targeted LNPs.

FIG. 9 illustrates the Huntingtin (HTT) gene knock-down in Huntingtin disease (HD) patient lymphocytes. IGF1R=IGF1R-sdAb.

FIG. 10 illustrates antibody concentration/levels detected with mass spectrometry on FC5 conjugated LNPs compared to nontargeted LNPs.

FIG. 11A illustrates the in vivo brain delivery of IGF1R-sdAb-ASO-LNP-Cy7 by optical imaging. Improved brain targeting is achieved with higher density of IGF1R-sdAb. IGF1R=IGF1R-sdAb.

FIG. 11B illustrates the ex-vivo brain delivery of IGF1R-sdAb-ASO-LNP-Cy7 by optical imaging.

FIG. 12A illustrates the in vivo distribution of IGF1R sdAb brain targeted vs non-targeted LNPs after a tail vein injection.

FIG. 12B illustrates the ex vivo brain distribution of IGF1R sdAb brain targeted vs non-targeted LNPs after a tail vein injection.

FIG. 12C illustrates the ex vivo brain slice distribution of IGF1R sdAb brain targeted vs non-targeted LNPs after a tail vein injection.

FIG. 13 illustrates the in vivo optical imaging of ASO-IR700 encapsulated in LNP targeted with A20 (top panel) or FC5 (bottom panel) over 16 hrs,

FIG. 14 illustrates the ex vivo brain and spine optical images of ASO-IR700 encapulated in LNPs targeted with A20 (left panel) and FC5 (right panel) at 16 hr after intravenous injection.

FIG. 15 illustrates the in vivo optical imaging of NIR815-lipid contained within LNP targeted with A20 (top panel) or FC5 (bottom panel) over 16 hrs,

FIG. 16 illustrates the ex vivo brain and spine optical images of NIR815-lipid contained within LNP targeted with A20 (left panel) and FC5 (right panel) at 16 hr after intravenous injection.

FIG. 17 illustrates near-infrared fluorescence imaging of ex vivo brain slices of mice after intracarotid injection of IGF1R-ASO-LNPs.

FIG. 18 illustrates the delivery and expression of fLuc mRNA in mouse head at 4, 12 and 24 hrs following intravenous injection of FC5-mRNA-LNPs.

FIG. 19 illustrates the quantification of head kinetics of delivery and expression of fLuc mRNA at 4, 11, 24 and 96 hrs following intravenous injection of FC5-mRNA-LNPs.

FIG. 20 illustrates the dose response delivery and expression of fLuc mRNA in mouse head following intravenous injection of FC5-mRNA-LNPs containing 25, 50 or 100 μg of mRNA.

FIG. 21 illustrates the quantification of delivery and expression of fLuc mRNA in mouse head following intravenous injection of FC5-mRNA-LNPs containing 25, 50 or 100 μg fLuc mRNA.

FIG. 22 illustrates the in vivo whole body (left), ex-vivo liver (middle) and ex-vivo brain (right) optical image at 4 hrs post IV injection of LNPs containing ASO fluorescently labeled with IR700 containing either 2.5% DMG-PEG or 2.5% DSG-PEG.

FIG. 23A illustrates the in vivo head analysis of optical images of NIR-815 lipid fluorescence at 4 hrs after IV injection of FC5 targeted ASO-LNPs containing either 2.5% DMG-PEG or 2.5% DSG-PEG in the mouse brain.

FIG. 23B illustrates the in vivo head analysis of optical images of ASO-IR700 fluorescence at 4 hrs after IV injection of FC5 targeted ASO-LNPs containing either 2.5% DMG-PEG or 2.5% DSG-PEG in the mouse brain.

FIG. 23C illustrates the ex vivo brain analysis of optical images of NIR-815 lipid fluorescence at 4 hrs after IV injection of FC5 targeted ASO-LNPs containing either 2.5% DMG-PEG or 2.5% DSG-PEG in the mouse brain.

FIG. 23D illustrates the ex vivo brain analysis of optical images of ASO-IR700 fluorescence at 4 hrs after IV injection of FC5 targeted ASO-LNPs containing either 2.5% DMG-PEG or 2.5% DSG-PEG in the mouse brain.

FIG. 24 illustrates the ex vivo organ analysis of optical images of NIR815 fluorescence at 4 hrs after IV injection of FC5 targeted ASO-LNPs containing either 2.5% DMG-PEG or 2.5% DSG-PEG in various tissues.

FIG. 25 illustrates the in vivo (right) optical images or region of interest head analysis (left) of A20- or FC5-targeted Fluc mRNA containing LNPs at 11 hrs after intravenous injection.

FIG. 26 illustrates the ex vivo brain optical images (left), ex-vivo whole brain region of interested analysis (middle) or ex vivo whole brain homogenate (right) of A20- or FC5-targeted Fluc mRNA containing LNPs at 11 hrs after intravenous injection.

FIG. 27 illustrates the in vivo whole body optical images of A20-(top panels) or FC5 (bottom panels)-targeted CRE recombinase mRNA containing LNPs at various days after intravenous injection.

FIG. 28 illustrates the in vivo head body optical images of A20-(left panels) or FC5 (right panels)-targeted CRE recombinase mRNA containing LNPs at various days after intravenous injection.

FIG. 29 illustrates the in vivo head body (left graph) and liver (right graph) analysis of A20- or FC5-targeted CRE recombinase mRNA containing LNPs at various days after intravenous injection.

FIG. 30 illustrates tdTomato fluorescent protein expression in naïve (top panels) and FC5-targeted CRE recombinase mRNA (bottom panels) injected Ai9 transgenic mice brain cortex sections. Mice express increased tdTomato fluorescence following Cre-mediated recombination.

It will be noted that throughout the appended drawings, like features are identified by like reference numerals.

### DETAILED DESCRIPTION

The present invention is directed to a technology for the site-specific conjugation of a lipid nanoparticle operable to encapsulate a therapeutic agent, such as a drug, a protein, an enzyme, or a nucleic acid, for example, to an antibody or antigen-binding fragment operable to transmigrate the blood-brain barrier (BBB), operably linked to the external surface of the lipid nanoparticle.

In embodiment, there is disclosed a pharmaceutical composition comprising:
a) a lipid nanoparticle operable to encapsulate a therapeutic agent, comprising a core and an external surface, the therapeutic agent being encapsulated within the core; the lipid nanoparticle having
   a size of the lipid nanoparticle of from about 30 to about 80 nm, or
   a pegylated lipid comprising a distearoyl-rac-glycerol (DSG)-PEG, a 1,2-distearoyl-sn-glycero-3-phosphoethanolamine-N-(DSPE)-PEG-DBCO, or a combination thereof, or
   a combination of: a size of from about 30 to about 80 nm and a pegylated lipid comprising a DSG-PEG, DSPE-PEG-DBCO or a combination thereof; and
b) an antibody or antigen binding fragment thereof operable to transmigrate the blood-brain barrier (BBB), wherein the antibody or antigen binding fragment thereof comprises complementarity determining regions (CDR1, CDR2 and CDR3), operably linked to the external surface of the lipid nanoparticle.

In embodiments, the size of the lipid nanoparticle may be from about 30 to about 80 nm, or about 40 to about 80 nm, or about 50 to about 80 nm, or about 60 to about 80 nm, or about 70 to about 80 nm, or about 30 to about 70 nm, or about 40 to about 70 nm, or about 50 to about 70 nm, or about 60 to about 70 nm, or about 30 to about 60 nm, or about 40 to about 60 nm, or about 50 to about 60 nm, or about 30 to about 50 nm, or about 40 to about 50 nm, or about 30 to about 40 nm, and preferably from about 40 to about 60 nm.

According to an embodiment, the lipid nanoparticle may comprise an ionizable cationic lipid, a helper phospholipid, cholesterol, a PEGylated lipid and combinations thereof.

According to an embodiment, the pegylated lipid comprises a distearoyl-rac-glycerol (DSG)-PEG and/or a 1,2-distearoyl-sn-glycero-3-phosphoethanolamine-N-(DSPE)-PEG. In addition to these, the pegylated lipid may further comprise a 1,2-dimyristoyl-rac-glycero-3-methoxy (DMG)-PEG, a 1,2-dipalmitoyl-sn-glycero-3-phosphoethanolamine-N(DPPE)-PEG.

In embodiments, the pegylated lipid may comprise a PEG group having a molecular weight of about 500 to about 5000 g/mol, preferably a molecular weight of about 2000 g/mol. For example, the DSG-PEG may be DSG-PEG₂₀₀₀. For example, the DSPE-PEG may be DSPE-PEG₂₀₀₀. For example, the DMG-PEG or the DSPE-PEG may be DMG-PEG₂₀₀₀ and DPPE-PEG₂₀₀₀, respectively.

In embodiments, the lipid nanoparticle may comprise a molar ratio of from about 1% to about 10%, or from about 2.5% to about 10%, or from about 5% to about 10%, or from about 1% to about 5%, or from about 2.5% to about 5%, or from about 1% to about 2.5% of DPPE-PEG, DSG-PEG, DMG-PEG, or DSPE-PEG, or combinations thereof.

In embodiments, the lipid nanoparticle may comprise a molar ratio of from about 1% to about 10%, or from about 2.5% to about 10%, or from about 5% to about 10%, or from about 1% to about 5%, or from about 2.5% to about 5%, or from about 1% to about 2.5% of DPPE-PEG₂₀₀₀, DSG-PEG₂₀₀₀, DMG-PEG₂₀₀₀, DSPE-PEG₂₀₀₀, or combinations thereof.

According to embodiments, the ionizable cationic lipid may be (6Z,9Z,28Z,31Z)-Heptatriaconta-6,9,28,31-tetraen-19-yl 4-(dimethylamino) butanoate (DLin-MC3-DMA), 2-[2,2-bis[(9Z,12Z)-octadeca-9,12-dienyl]-1,3-dioxolan-4-yl]-N,N-dimethylethanamine (DLin-KC2-DMA), [(4-hydroxybutyl)azanediyl]di(hexane-6,1-diyl)bis(2-hexyldecanoate) (ALC-0315), Heptadecan-9-yl 8-{(2-hydroxyethyl)[6-oxo-6-(undecyloxy)hexyl]amino}octanoate (SM-102), and combinations thereof.

In embodiments, the lipid nanoparticle may comprise a molar ratio of from about 10% to about 60%, or from about 10% to about 50%, or from about 10% to about 40%, or from about 10% to about 30%, or from about 10% to about 20%, or 20% to about 60%, or from about 20% to about 50%, or from about 20% to about 40%, or from about 20% to about 30%, or 30% to about 60%, or from about 30% to about 50%, or from about 30% to about 40%, or 40% to about 60%, or from about 40% to about 50%, or 50% to about 60% of an ionizable cationic lipid, and preferably from about 40% to about 50% of an ionizable cationic lipid.

The helper phospholipid may be 1,2-distearoyl-sn-glycero-3-phosphocholine (DSPC), 1,2-dioleoyl-sn-glycero-3-phosphocholine (DOPC), 1,2-Dioleoyl-sn-glycero-3-phosphoethanolamine (DOPE), 1-stearoyl-2-oleoyl-sn-glycero-3-phosphocholine (SOPC) and combinations thereof.

In embodiments, the lipid nanoparticle may comprise a molar ratio of from about 2% to about 20%, or from about 5% to about 20%, or from about 10% to about 20%, or from about 15% to about 20%, or from about 2% to about 15%, or from about 5% to about 15%, or from about 10% to about 15%, or from about 2% to about 10%, or from about 5% to about 10%, or from about 2% to about 5%, and preferably from about 5% to about 10% of DSPC, DOPC, DOPE, and SOPC.

In embodiments, the lipid nanoparticle may comprise a molar ratio of from about 20% to about 50%, or from about 30% to about 50%, or from about 35% to about 50%, or from about 40% to about 50%, or from about 20% to about 40%, or from about 30% to about 40%, or from about 35% to about 40%, or from about 20% to about 35%, or from about 30% to about 35%, or from about 20% to about 30%, and preferably from about 35% to 40% of cholesterol.

The invention also encompasses an antibody or an antigen-binding fragment thereof, operable to transmigrate the BBB which is operably linked to the external surface of the lipid nanoparticle. For example, according to an embodiment, the antibody or antigen binding fragment thereof operable to transmigrate the BBB comprises a cysteine amino acid operable to make a thioether covalent bond, and/or an epsilon amino group operable to make an amide covalent bond, for conjugation with the lipid nanoparticle. According to another embodiment, the antibody or antigen binding fragment thereof, operable to transmigrate the BBB may comprise a reactive functional group for conjugation with the lipid nanoparticle. For example, the reactive functional group is an azido group.

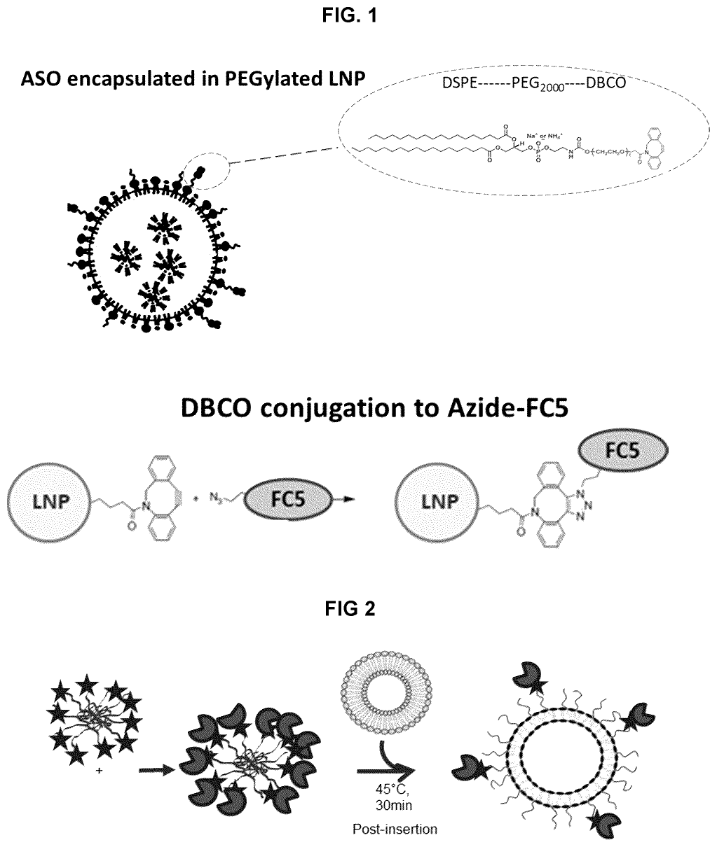

Now referring to FIG. 1, according to another embodiment, and which illustrates a general strategy of the present invention. FIG. 1 illustrates an embodiment where the lipid nanoparticle (LNP), particularly a pegylated lipid nanoparticle, is conjugated with sdAbs. In this example, DSPE-PEG₂₀₀₀-DBCO is conjugated to a sequon-linked O-glycan comprising an azido group, which “clicks” the C-terminus of sdAbs and the LNP. The C-terminus of sdAbs is located away from complementarity-determining regions (CDRs) which allows the introduction of an O-glycosylation site (a sequon) without interfering with target antigen recognition and affinity.

In embodiment, there is disclosed an antibody or antigen-binding fragment that is operable to transmigrate the blood-brain barrier (BBB), wherein the antibody or antigen-binding fragment comprises complementarity determining regions (CDR1, CDR2 and CDR3) and an added O-glycosylation sequon glycosylated with an O-glycan. In embodiment, the added O-glycosylation sequon is glycosylated with an O-glycan of general formula (I):

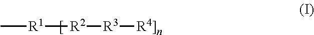

wherein
R¹ is an initial N-acetylgalactosamine (GalNAc);
n=1, or 2;
R² are each independently absent, or galactose (Gal), GalNAc, N-Acetylglucosamine (GlcNAc), or a sialic acid;
R³ are each independently absent, Gal or a sialic acid;
R⁴ are each independently absent or a sialic acid.

Therefore, according to an embodiment, the pharmaceutical composition of the present invention may comprise an antibody or antigen-binding fragment operable to transmigrate the blood-brain barrier (BBB), which comprises such an added O-glycosylation sequon glycosylated with an O-glycan. The O-glycan may comprise a reactive functional group, i.e, a 9N3-Neu5Ac moiety, which is to be conjugated with a functional moiety comprising a functionalized cyclooctyne—in this case, the lipid nanoparticle encapsulating a therapeutic agent. Upon conjugation this provides a pharmaceutical composition comprising an antibody or antigen-binding fragment conjugated to the functional moiety (i.e., the lipid nanoparticle) operably linked to the O-glycan.

The functionalized cyclooctyne may be dibenzocyclooctyne (DBCO), bicyclononyne (BCN), cyclooctyne (COT), monofluorinated cyclooctyne (MOFO), difluorocyclooctyne (DIFO), dimethoxyazacyclooctyne (DIMAC), dibenzoazacyclooctyne (DIBAC), biarylazacyclooctynone (BARAC), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), or combinations thereof.

In embodiments, the lipid nanoparticle may comprises a molar ratio of from about 0.05% to about 2%, or from about 0.05% to about 1.5%, or from about 0.05% to about 1.0%, or from about 0.05% to about 0.5%, or from about 0.05% to about 0.1%, or from about 0.1% to about 2%, or from about 0.1% to about 1.5%, or from about 0.1% to about 1.0%, or from about 0.1% to about 0.5%, or from about 0.5% to about 2%, or from about 0.5% to about 1.5%, or from about 0.5% to about 1.0%, or from about 1% to about 2%, or from about 1% to about 1.5%, or from about 1.5% to about 2%, and preferably from about 0.05% to about 1% of a pegylated lipid functionalized with a cyclooctyne.

In embodiments, the lipid nanoparticle may comprises a molar ratio of from about 0.05% to about 2%, or from about 0.05% to about 1.5%, or from about 0.05% to about 1.0%, or from about 0.05% to about 0.5%, or from about 0.05% to about 0.1%, or from about 0.1% to about 2%, or from about 0.1% to about 1.5%, or from about 0.1% to about 1.0%, or from about 0.1% to about 0.5%, or from about 0.5% to about 2%, or from about 0.5% to about 1.5%, or from about 0.5% to about 1.0%, or from about 1% to about 2%, or from about 1% to about 1.5%, or from about 1.5% to about 2%, and preferably from about 0.05% to about 1% of DSG-PEG₂₀₀₀-DBCO, DSPE-PEG₂₀₀₀-DBCO, DMG-PEG₂₀₀₀-DBCO, DPPE-PEG₂₀₀₀-DBCO, or combinations thereof.

In embodiments, the lipid nanoparticle may comprise, for example a molar ratio of from about 40% to about 50% DLin-MC3-DMA, ALC-0315, or a combination thereof; from about 5% to about 10% DSPC; from about 35% to about 40% cholesterol; from about 1% to about 5% DSG-PEG₂₀₀₀, DSPE-PEG₂₀₀₀, DMG-PEG₂₀₀₀, or DPPE-PEG₂₀₀₀, or combinations thereof; and from about 0.05% to about 2% of DSPE-PEG₂₀₀₀-DBCO.

In embodiment, there is disclosed an antibody or antigen-binding fragment that is operable to transmigrate the blood-brain barrier (BBB), wherein the antibody or antigen-binding fragment comprises complementarity determining regions (CDR1, CDR2 and CDR3) and an added O-glycosylation sequon glycosylated with an O-glycan. In embodiment, the added O-glycosylation sequon is glycosylated with an O-glycan of general formula (I):

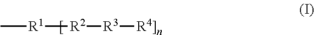

wherein
R¹ is an initial N-acetylgalactosamine (GalNAc);
n=1, or 2;
R² are each independently absent, or galactose (Gal), GalNAc, N-Acetylglucosamine (GlcNAc), or a sialic acid;
R³ are each independently absent, Gal or a sialic acid;
R⁴ are each independently absent or a sialic acid.

According to an embodiment of the present invention, the antibody or antigen-binding fragment comprises an added O-glycosylation sequon, to be glycosylated with an O-glycan. As used herein, the term “sequon” refers to the sequence of amino acids required for glycosylation, in this instant case, O-glycosylation. Proteins, antibodies and antigen-binding fragment included may comprise naturally occurring sequons. Therefore, as used herein, the sequon comprised in the invention is an added O-glycosylation sequon, found in addition to any other sequon that may be present in the antibody or antigen-binding fragment of the invention.

In embodiments, the added O-glycosylation sequon may comprise an amino acid sequence comprising PTTDSTX₁PAPTTK, where X₁ is S or T (SEQ ID NO: 1); FFPX₂PGP, where X₂ is S or T (SEQ ID NO: 2); GVGVX₃ETP, where X₃ is S or T (SEQ ID NO: 3); AAAX₄PAP, where X₄ is S or T (SEQ ID NO: 4); and APALQPX₅QGAMPA, where X₅ is S or T (SEQ ID NO: 5), or combinations thereof.

In embodiments, the added O-glycosylation sequon may comprise an amino acid sequence comprising PTTDSTTPAPTTK (SEQ ID NO: 6), PTTDSTSPAPTTK (SEQ ID NO: 7), FFPTPGP (SEQ ID NO: 8); FFPSPGP (SEQ ID NO: 9), GVGVTETP (SEQ ID NO: 10), GVGVSETP (SEQ ID NO: 11), AAATPAP (SEQ ID NO: 12), AAASPAP (SEQ ID NO: 13); APALQPTQGAMPA (SEQ ID NO: 14), and APALQPSQGAMPA (SEQ ID NO: 15).

Different O-sequons may be useful in the instant invention and they are listed in Table 1.

TABLE 1
Sequons

| SEQ ID NO: | Sequence | Description |
|---|---|---|
| SEQ ID NO: 6 | PTTDSTTPAPTTK | Sequon EA2: aa 153-165 of rat Muc10-based |
| | | on sequon naturally present in mucin |
| SEQ ID NO: 8 | FFPTPGP | a synthetic sequon based on the work |
| | | published by Gerken et al., 2006 to optimize |
| | | in vitro O-glycosylation |
| SEQ ID NO: 10 | GVGVTETP | Sequon from interferon alpha 2 |
| SEQ ID NO: 12 | AAATPAP | shortest sequence motif for high level mucin- |
| | | type O-glycosylation based on Yoshida et al., |
| | | 1997 |
| SEQ ID NO: 14 | APALQPTQGAMPA | O-linked sequon present in G-CSF |
| SEQ ID NO: 25 | SSFFPTPGP | Sequon OG1: SS linker and sequon of SEQ ID |
| | | NO: 8 |
| SEQ ID NO: 26 | FFPTPGPFFPTPGP | Two sequons of SEQ ID NO: 8 in tandem |
| SEQ ID NO: 27 | SSGGGFFPTPGP | Sequon OG2: SSGGG linker and sequon of |
| | | SEQ ID NO: 8 |
| SEQ ID NO: 28 | SSGGGFFPTPGPFFPTPGP | Sequon OG3: SSGGG linker and two sequons |
| | | of SEQ ID NO: 8 in tandem |
| SEQ ID NO: 29 | SSGGGGVGVTETP | Sequon OG4: SSGGG linker and sequon of |
| | | SEQ ID NO: 10 (sequon from interferon alpha 2) |
| SEQ ID NO: 30 | GGGGAPALQPTQGAMPA | Sequon OG6: GGGG linker and sequon of SEQ |
| | | ID NO: 14 (O-linked sequon present in G-CSF) |
| SEQ ID NO:31 | SSGGGAAATPAP | Sequon OG5: SSGGG linker and sequon of |
| | | SEQ ID NO: 12 (shortest sequence motif for |
| | | high level mucin-type O-glycosylation based on |
| | | Yoshida et al., 1997) |

In embodiment, the added O-glycosylation sequon may be at a C-terminus of the antibody or antigen-binding fragment. According to another embodiment, the O-glycosylation sequon may be at the N-terminus of the antibody or antigen-binding fragment, within the sequence of the antibody or antigen-binding fragment, at the C-terminus of the antibody or antigen-binding fragment, or combinations thereof.

In embodiment, as detailed above, the added O-glycosylation sequon is glycosylated with an O-glycan of general formula (I):

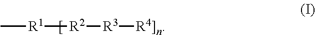

In embodiments, the R¹ is an initial N-acetylgalactosamine (GalNAc), where n=1, or 2. R² may each independently be absent, or galactose (Gal), GalNAc, N-Acetylglucosamine (GlcNAc), or a sialic acid. R³ may each independently be absent, Gal or a sialic acid. R⁴ may each independently be absent or a sialic acid. According to another embodiment, n may be equal to 1 and R² may be Gal. According to yet another embodiment, the R³ may be a sialic acid selected from the group consisting of Neu5Ac and 9N3-Neu5Ac, and R⁴ may be absent.

In embodiment, the initial GalNAc, and/or any one of the R¹, R², R³ and R⁴ may be further modified with one or more pharmaceutical composition. For example, the pharmaceutical composition may be one or more of a methyl group, an acetyl group, a sulfate group, or a combination thereof.

According to an embodiment, the O-glycan may have the general formula (II):

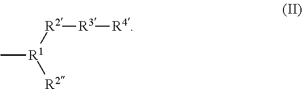

In the general formula (II), the R^{2′} may be Gal, or GlcNAc. The R^{3′} may be Gal or a sialic acid. The R^{4′} may be absent, or a sialic acid. The R^{2″} may be GlcNAc or a sialic acid.

According to another embodiment, the R^{2′} may be Gal, the R^{3′} may be a sialic acid consisting of Neu5Ac, and the R^{4′} may be absent or a sialic acid consisting of 9N₃-Neu5Ac; and the R^{2″} may be a sialic acid consisting of Neu5Ac.

In embodiments, sialic acids are a class of alpha-keto acid sugars with a nine-carbon backbone found widely distributed in animal tissues and related forms are found to a lesser extent in other organisms like in some micro-algae, bacteria and archaea. Sialic acids are commonly part of glycoproteins, glycolipids or gangliosides, where they decorate the end of sugar chains at the surface of cells or soluble proteins. According to embodiments of the present invention, the sialic acid may be N-Acetylneuraminic acid (Neu5Ac), 9-azido-N-Acetylneuraminic acid (9N3-Neu5Ac), N-azidoacetylneuraminic acid (Neu5NAz), or a combination thereof.

According to an embodiment of the present invention, the antibody or antigen-binding fragment may be a single-domain antibody (sdAb), a fragment antigen-binding (Fab), a single-chain variable fragment (scFv), or a single-chain fragment antigen-binding (scFab). The antibody or antigen-binding fragment may be an IgA, an IgD, an IgE, an IgG, or an IgM. In embodiments, the antibody or an antigen-binding fragment that specifically binds to a target antigen comprises four framework regions (FR1 to FR4) and three complementarity determining regions (CDR1, CDR2 and CDR3).

As used herein, the expression “substantially identical sequence” is intended to mean an amino acid sequence which may comprise one or more conservative amino acid mutations. It is known in the art that the introduction of one or more conservative amino acid mutations to a reference sequence may yield a mutant peptide with no substantial change in physiological, chemical, physico-chemical or functional properties compared to the reference sequence. In such a case, the reference and mutant sequences would be considered “substantially identical” polypeptides. A conservative amino acid substitution is defined herein as the substitution of an amino acid residue for another amino acid residue with similar chemical properties (e.g., size, charge, or polarity). According to one embodiment, one or more conservative amino acid mutations may be made to the one or more framework regions of the sdAb while maintaining both the CDR sequences and the overall structure of the CDR of the antibody or antigen-binding fragment; thus the specificity and binding of the antibody or antigen-binding fragment are maintained. According to another embodiment, one or more conservative amino acid mutations may be made to the one or more framework regions of the sdAb and to a CDR sequence while maintaining the antigen-binding function of the overall structure of the CDR of the antibody or antigen-binding fragment; thus the specificity and binding of the antibody or antigen-binding fragment are maintained.

In a non-limiting example, a conservative mutation may be a conservative amino acid substitution. Such a conservative amino acid substitution may substitute a basic, neutral, hydrophobic, or acidic amino acid for another amino acid of the same group. By the term “basic amino acid” it is meant a hydrophilic amino acid having a side chain pK value of greater than 7, which is typically positively charged at physiological pH. Basic amino acids include histidine (His or H), arginine (Arg or R), and lysine (Lys or K). By the term “neutral amino acid” (also “polar amino acid”), it is meant a hydrophilic amino acid having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). By the term “hydrophobic amino acid” (also “non-polar amino acid”) it is meant an amino acid exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G). “Acidic amino acid” refers to a hydrophilic amino acid having a side chain pK value of less than 7, which is typically negatively charged at physiological pH. Acidic amino acids include glutamate (Glu or E) and aspartate (Asp or D).

Sequence identity is used to evaluate the similarity of two sequences. It is determined by calculating the percentage of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 90% identical; in another example, the substantially identical sequences may be at least 90%, at least 91%, at least 92%, at least 93%, at least 94%, at least 95%, at least 96%, at least 97%, at least 98%, or at least 99% identical, or any percentage there between, at the amino acid level to sequences described herein. Importantly, a substantially identical sequence retains the activity and specificity of the reference sequence. In a non-limiting embodiment, the difference in sequence identity may be due to one or more conservative amino acid mutations. In a non-limiting example, the present invention may be directed to an antibody or antigen-binding fragment comprising a sequence at least at least 95%, at least 98%, or at least 99% identical to that of one or more of the antibodies or antigen-binding fragments described herein.

The antibody or an antigen-binding fragment of the present invention is operable to transmigrate the blood-brain barrier (BBB). As used herein, the expression “operable to transmigrate the blood-brain barrier (BBB)” is intended to mean that the antibody or antigen-binding fragment of the present invention is capable of transmigration across the blood brain barrier. The brain is separated from the rest of the body by a specialized endothelial tissue known as the blood-brain barrier (BBB). The endothelial cells of the BBB are connected by tight junctions and efficiently prevent many therapeutic pharmaceutical compositions from entering the brain. In addition to low rates of vesicular transport, one specific feature of the BBB is the existence of enzymatic barrier(s) and high level(s) of expression of ATP-dependent transporters on the abluminal (brain) side of the BBB, including P-glycoprotein (Gottesman and Pastan, 1993; Watanabe et al., 1995), which actively transport various molecules from the brain into the blood stream (Samuels et al., 1993). Only small (<500 Daltons) and hydrophobic (Pardridge, 1995) molecules can more readily cross the BBB. Thus, the ability of the antibody or fragment thereof as described above to specifically bind the surface receptor, internalize into brain endothelial cells, and undergo transcytosis across the BBB by evading lysosomal degradation is useful in the neurological field.

The term “antibody”, also referred to in the art as “immunoglobulin” (Ig), as used herein refers to an antigen-binding protein constructed from paired heavy and light polypeptide chains; various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (V_{L}) and a constant (C_{L}) domain, while the heavy chain folds into a variable (V_{H}) and three constant (C_{H}1, C_{H}2, C_{H}3) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding a target antigen and can therefore show significant sequence diversity between antibodies. The constant regions show less sequence diversity, and are responsible for binding a number of natural proteins to elicit important biochemical events. The variable region of an antibody contains the antigen-binding determinants of the molecule, and thus determines the specificity of an antibody for its target antigen. The majority of sequence variability occurs in six hypervariable regions, three each per variable heavy (V_{H}) and light (V_{L}) chain; the hypervariable regions combine to form the antigen-binding site, and contribute to binding and recognition of an antigenic determinant. The specificity and affinity of an antibody for its antigen is determined by the structure of the hypervariable regions, as well as their size, shape, and chemistry of the surface they present to the antigen. Various schemes exist for identification of the regions of hypervariability, the two most common being those of Kabat and of Chothia and Lesk. Kabat and Wu (1991) define the “complementarity-determining regions” (CDRs) based on sequence variability at the antigen-binding regions of the V_{H} and V_{L} domains. Chothia and Lesk (1987) define the “hypervariable loops” (H or L) based on the location of the structural loop regions in the V_{H} and V_{L} domains. These individual schemes define CDR and hypervariable loop regions that are adjacent or overlapping. Those of skill in the antibody art often utilize the terms “CDR” and “hypervariable loop” interchangeably, and they may be so used herein. The CDRs/loops are identified herein according to the IMGT nomenclature scheme (i.e., CDR1, 2 and 3, for each variable region).

An “antibody fragment” or “antigen-binding fragment” as referred to herein may include any suitable antigen-binding antibody fragment known in the art. The antibody fragment may be a naturally-occurring antibody fragment, or it may be a non-naturally occurring antibody fragment obtained, for example, by manipulation of a naturally-occurring antibody or by recombinant methods. For example, an antibody fragment may include, but is not limited to, a Fv, a single-chain Fv (scFv; a molecule consisting of V_{L} and V_{H} connected with a peptide linker), a Fab, a F(ab′)₂, single-domain antibody (sdAb; a fragment composed of a single V_{L} or V_{H} or a V_{H}H), or a multivalent presentation of any of these. Antibody fragments such as those just described may require one or more linker sequences, disulfide bonds, or other type of covalent bond to link different portions of the fragments; those of skill in the art will be familiar with the requirements of the different types of fragments and various approaches for their construction.

In a non-limiting example, the antigen-binding fragment of the present invention may be a sdAb derived from a naturally-occurring source. Heavy chain antibodies of camelid origin (Hamers-Casterman et al, 1993) lack light chains and thus their antigen binding sites consist of one domain, termed V_{H}H. SdAbs have also been observed in shark and are termed V_{NAR} (Nuttall et al, 2003). Other sdAbs may be engineered based on human Ig heavy and light chain sequences (Jespers et al, 2004; To et al, 2005). As used herein, the term “sdAb” includes an sdAb directly isolated from a V_{H}, V_{H}H, V_{L}, or V_{NAR} reservoir of any origin through phage display or other technology, an sdAb derived from the aforementioned sdAb, a recombinantly produced sdAb, as well as an sdAb generated through further modification of such sdAb by humanization, affinity maturation, stabilization, solubilization, camelization, or other methods of antibody engineering. Also encompassed by the present invention are homologues, derivatives, or fragments that retain the antigen-binding function and specificity of the sdAb.

SdAbs possess desirable properties for antibody molecules, such as high thermostability, high detergent resistance, relatively high resistance to proteases (Dumoulin et al, 2002) and high production yield (Arbabi-Ghahroudi et al, 1997). They can also be engineered to have very high affinity by isolation from an immune library (Li et al, 2009) or by in vitro affinity maturation (Davies & Riechmann, 1996). Further modifications to increase stability, such as the introduction of one or more non-canonical disulfide bonds (Hussack et al, 2011a,b; Kim et al, 2012), may also be brought to the sdAb.

A person of skill in the art would be well-acquainted with the structure of a single-domain antibody (see, for example, 3DWT, 2P42 in Protein Data Bank). An sdAb comprises a single immunoglobulin domain that retains the immunoglobulin fold; most notably, only three CDR/hypervariable loops form the antigen-binding site. However, and as would be understood by those of skill in the art, not all CDRs may be required for binding the antigen. For example, and without wishing to be limiting, one, two, or three of the CDRs may contribute to binding and recognition of the antigen by the sdAb of the present invention. The CDRs of the sdAb or variable domain are referred to herein as CDR1, CDR2, and CDR3.

The present invention further encompasses an antibody or an antigen-binding fragment that is “humanized” using any suitable method known in the art, such as, but not limited to, CDR grafting or veneering. Humanization of an antibody or an antigen-binding fragment comprises replacing an amino acid in the antibody or antigen-binding fragment sequence with its human counterpart, as found in the human consensus sequence, without substantial loss of antigen-binding ability or specificity; this approach reduces immunogenicity of the antibody or antigen-binding fragment when introduced into a human subject. In the process of CDR grafting, one or more than one of the CDRs defined herein may be fused or grafted to a human variable region (V_{H}, or V_{L}), to other human antibody (IgA, IgD, IgE, IgG, and IgM), to a human antibody fragment framework region (Fv, scFv, Fab) or to another protein of similar size and nature onto which a CDR can be grafted (Nicaise et al, 2004). In such a case, the conformation of the one or more than one hypervariable loop is likely preserved, and the affinity and specificity of the antibody or antigen-binding fragment for its target (i.e., a target antigen) is likely minimally affected. CDR grafting is known in the art and is described in at least the following: U.S. Pat. Nos. 6,180,370, 5,693,761, 6,054,297, 5,859,205, and European Patent No. 626390. Veneering, also referred to in the art as “variable region resurfacing”, involves humanizing solvent-exposed positions of an antibody or antigen-binding fragment; thus, preserving buried non-humanized residues, which may be important for CDR conformation, while minimizing the potential for immunological reaction against solvent-exposed regions. Veneering is known in the art and is described in at least the following: U.S. Pat. Nos. 5,869,619, 5,766,886, 5,821,123, and European Patent No. 519596. Persons of skill in the art would also be amply familiar with methods of preparing such humanized antibody fragments and humanizing amino acid positions.

The antibody or antigen-binding fragment according to the present invention may comprise one or more additional sequences to aid in expression, detection or purification of the antibody or antigen-binding fragment. Any such sequence or tag known to those of skill in the art may be used. For example, and without wishing to be limiting, the antibody or antigen-binding fragment may comprise a targeting or signal sequence (such as, but not limited to, ompA or pelB), a detection/purification tag (such as, but not limited to, c-Myc, HA, His5, or His6), or a combination of any two or more thereof. In another example, the additional sequence may be a biotin recognition site such as that described in WO/1995/004069 or by Voges et al. in WO/2004/076670. As is also known to those of skill in the art, a linker sequence may be used in conjunction with the additional sequence or tag, or may serve as a detection/purification tag.

According to an embodiment, there is disclosed an antibody or antigen-binding fragment according to the present invention, linked to a functional moiety, optionally by a linker sequence. For example, and according to an embodiment, the added O-glycosylation sequon may be linked to a first functional moiety via a peptide linker, or another portion of the antibody or antigen-binding fragment may be functionally linked to a first functional moiety via a peptide linker.

In another embodiment, there is disclosed a pharmaceutical composition comprising an antibody or antigen-binding fragment according to the present invention, linked to a functional moiety, optionally by a linker sequence. According to another embodiment, there is disclosed a pharmaceutical composition comprising an antibody or antigen-binding fragment according to the present invention, comprising a functional moiety operably linked to the O-glycan.

In embodiments of the antibody or antigen-binding fragment according to the present invention, or of the pharmaceutical composition, the antibody or antigen-binding fragment may be linked to the functional moiety via a linker (also known as a linker sequence). As used herein, the term “linker sequence” is intended to mean a short (typically 40 amino acids or fewer) peptide sequence that is introduced between protein domains. Linker sequences are often composed of flexible residues such as glycine and serine so that the linked protein domains are free to move relative to one another. The linker sequence can be any linker sequence known in the art that would allow for the antibody and the functional moiety of the present invention to be operably linked for the desired function. The linker may be any sequence known in the art (either a natural or synthetic linker) that allows for an operable fusion comprising an antibody or antigen-binding fragment linked to a polypeptide (e.g., the functional moiety). For example, the linker sequence may be a linker sequence L such as (SS)ₙ, (GGG)ₙ, (GGGG)ₙ, (GGGS)ₙ, (GGGGS)ₙ (SEQ ID NO: 23) or (SSGGG)ₙ (SEQ ID NO: 24), wherein n is equal to or greater than 1, or from about 1 to about 5, or from about 1 to 15; or n may be any number that would allow for the operability of the pharmaceutical composition of the present invention. In another example, the linker may be an amino acid sequence, for example, an amino acid sequence that comprises about 1 to about 40 amino acids, or about 3 to about 40 amino acids, or about 5 to about 40 amino acids, or about 10 to about 40 amino acids, or about 15 to about 40 amino acids, or about 20 to about 40 amino acids, or about 25 to about 40 amino acids, or about 30 to about 40 amino acids, or about 35 to about 40 amino acids, or about 3 to about 35 amino acids, or about 5 to about 35 amino acids, or about 10 to about 35 amino acids, or about 15 to about 35 amino acids, or about 20 to about 35 amino acids, or about 25 to about 35 amino acids, or about 30 to about 35 amino acids, or about 3 to about 30 amino acids, or about 5 to about 30 amino acids, or about 10 to about 30 amino acids, or about 15 to about 30 amino acids, or about 20 to about 30 amino acids, or about 25 to about 30 amino acids, or about 3 to about 25 amino acids, or about 5 to about 25 amino acids, or about 10 to about 25 amino acids, or about 15 to about 25 amino acids, or about 20 to about 25 amino acids, or about 3 to about 20 amino acids, or about 5 to about 20 amino acids, or about 10 to about 20 amino acids, or about 15 to about 20 amino acids, or about 3 to about 15 amino acids, or about 5 to about 15 amino acids, or about 10 to about 15 amino acids, or about 15 to about 20 amino acids, or about 3 to about 10 amino acids, or about 5 to about 10 amino acids, or about 3 to about 5 amino acids, or up to 3, up to 5, up to 10, up to 15, up to 20, up to 25, up to 30, up to 35, or up to 40 amino acids.

As used herein, the term “functional moiety” is intended to mean a part of the pharmaceutical composition having an activity, purpose, or task; relating to the way in which the pharmaceutical composition is intended to work or operate. As used herein, the term “functional moiety” is related to the generic term “payload” which is referred to above as the moiety of interest to be conjugated to the antibody or antigen-binding fragment via the O-glycan linked to the added sequon. According to preferred embodiments, the functional moiety may be a lipid nanoparticle as defined above, encapsulating a therapeutic agent.

In embodiments, the functional moiety may be linked to the antibody or antigen-binding fragment, for example, through a chemical link pursuant to a chemical reaction, through fusion of the antibody or antigen-binding fragment with the functional moiety, obtained for example using recombinant DNA technology, and/or conjugated to the antibody or antigen-binding fragment via the O-glycan linked to the added sequon.

According to an embodiment, the antibody or antigen-binding fragment of the pharmaceutical composition may also be fused to a peptide, a polypeptide (e.g. growth factor CIBP2, an antimicrobial cyclic peptide), a protein, an enzyme [such as iduronate-2-sulfatase (IDS), acid beta-glucosidase (GCase), a serine protease, a growth factor, etc.], another (or the same) antibody or a fragment operable to bind a target epitope (e.g., an anti-microbial antibody, an anti-inflammatory antibody, an intrabody, a BBB-crossing antibody, a neurodegeneration target antibody, an ion channel targeting antibody, a cancer associated antigen antibody, a checkpoint inhibitor targeting antibody, or a GPCR targeting antibody) (for any use and for example for use in imaging, diagnostic, affinity purification, etc.), or a combination of any two or more thereof, in which both the antibody or antigen-binding fragment and the rest of the pharmaceutical composition (i.e. the functional moiety) remain functional for their intended purpose. In a preferred embodiment, the pharmaceutical composition may be fused to a second antibody or antigen-binding fragment, operable to bind a target epitope, which may be the same as, or distinct from the epitope of the antibody or antigen-binding fragment of the present invention.

The antibody or antigen-binding fragment of the present invention may also be in a multivalent display format, also referred to herein as multivalent presentation. Multimerization may be achieved by any suitable method known in the art. For example, and without wishing to be limiting in any manner, multimerization may be achieved using self-assembly molecules such as those described in Zhang et al (2004a; 2004b) and WO2003/046560, where pentabodies are produced by expressing a fusion protein comprising the antibody or antigen-binding fragment of the present invention and the pentamerization domain of the B-subunit of an AB5 toxin family (Merritt & Hol, 1995). A multimer may also be formed using the multimerization domains described by Zhu et al. (2010); this form, referred to herein as a “combody” form, is a fusion of the antibody or fragment of the present invention with a coiled-coil peptide resulting in a multimeric molecule (Zhu et al., 2010). Other forms of multivalent display are also encompassed by the present invention. For example, and without wishing to be limiting, the antibody or antigen-binding fragment may be presented as a dimer, a trimer, or any other suitable oligomer. This may be achieved by methods known in the art (Spiess et al, 2015), for example by direct linking connection (Nielsen et al, 2000), c-jun/Fos interaction (de Kruif & Logtenberg, 1996), or “Knob into holes” interaction (Ridgway et al, 1996).

Another method known in the art for multimerization is to dimerize the antibody or antigen-binding fragment using an Fc domain, such as, but not limited to a human Fc domain. The Fc domain may be selected from various classes including, but not limited to, IgG, IgM, or various subclasses including, but not limited to IgG1, IgG2, etc. In this approach, the Fc gene is inserted into a vector along with the sdAb gene to generate a sdAb-Fc fusion protein (Bell et al, 2010; Iqbal et al, 2010); the fusion protein is recombinantly expressed, then purified. For example, and without wishing to be limiting in any manner, a multivalent display format may encompass a chimeric or humanized format of V_{H}H of the present invention linked to an Fc domain, or bi or tri-specific antibody fusions with two or three V_{H}Hs recognizing unique epitopes. Such antibodies are easy to engineer and produce, can greatly extend the serum half-life of a sdAb, and may be excellent tumor imaging reagents (Bell et al., 2010).

The Fc domain in the multimeric complex as just described may be any suitable Fc fragment known in the art. The Fc fragment may be from any suitable source; for example, the Fc fragment may be of mouse or human origin. In a specific, non-limiting example, the Fc fragment may be a mouse Fc2b fragment or a human Fc1 fragment (Bell et al, 2010; Iqbal et al, 2010). The Fc fragment may be fused to the N-terminal or C-terminal end of the V_{H}H or humanized version of the present invention.

Each subunit of the multimers described above may comprise the same or different antibodies or antigen-binding fragments of the present invention, which may have the same or different specificity. Additionally, the multimerization domains may be linked to the antibody or antigen-binding fragment using a linker, as required; such a linker should be of sufficient length and appropriate composition to provide flexible attachment of the two molecules but should not hamper the antigen-binding properties of the antibody or antigen-binding fragment. As defined above, the linker sequence can be any linker known in the art that would allow for the pharmaceutical composition of the present invention to be prepared and be operable for the desired function.

According to embodiments, the therapeutic agent may be a peptide, a polypeptide, a protein, an enzyme, an antibody, an antibody fragment, a nucleic acid, or combinations thereof. The nucleic acid may be for example an antisense oligonucleotide (ASO), a duplex RNA, a single stranded RNA molecule, a ministering DNA (msDNA), a DNA plasmid, or combinations thereof. The duplex RNA may be a small interfering RNA (siRNA), a microRNA (miRNA), or a combination thereof. The single stranded RNA molecule may be a short hairpin RNA (shRNA), an mRNA, and anti-miRNA, or combinations thereof.

According to another embodiment, the present invention also encompasses a composition comprising one or more than one pharmaceutical composition as described herein. The composition may comprise a single sdAb and/or pharmaceutical composition as described above, or the composition may comprise a mixture of sdAbs and/or pharmaceutical compositions. Furthermore, in a composition comprising a mixture of sdAbs and/or pharmaceutical compositions of the present invention, the sdAbs and/or pharmaceutical compositions may have the same specificity, or they may differ in their specificities.

A composition according to the invention may also comprise a pharmaceutically acceptable diluent, excipient, or carrier. The diluent, excipient, or carrier may be any suitable diluent, excipient, or carrier known in the art that is compatible with other ingredients in the composition, that is compatible with the method of delivery of the composition, and that is not deleterious to the recipient of the composition. The composition may be in any suitable form; for example, the composition may be provided in suspension form, powder form (such as, but not limited to, lyophilized or encapsulated), capsule form or tablet form. For example, and without wishing to be limiting, when the composition is provided in suspension form, the carrier may comprise water, saline, or a suitable buffer, and optionally comprise one or more additives to improve solubility and/or stability. Reconstitution to produce a suspension may be effected in a buffer at a suitable pH to ensure the viability of the antibody or antigen-binding fragment. Dry powders may also include additives to improve stability and/or carriers to increase bulk/volume; for example, and without wishing to be limiting, the dry powder composition may comprise sucrose or trehalose. In a specific, non-limiting example, the composition may be formulated for delivery of the antibody or antigen-binding fragment to the gastrointestinal tract of the subject. Thus, the composition may comprise encapsulation, time release, or other suitable technologies for delivery of the sdAb and/or pharmaceutical composition of the present invention. It would be within the competency of a person of skill in the art to prepare suitable compositions comprising the present sdAb and/or pharmaceutical composition.

According to another embodiment, there is disclosed a method of delivering a therapeutic agent across the BBB, comprising administering the pharmaceutical composition or a composition according to the present invention to a subject in need thereof, for the treatment of a brain disease, for example via gene silencing, addition or editing.

According to another embodiment, there is disclosed a use of a pharmaceutical composition or a composition according to the present invention, for the delivery of a therapeutic agent in the brain of a subject in need thereof, for the treatment of a brain disease, for example via gene silencing, addition or editing.

The present invention will be more readily understood by referring to the following examples which are given to illustrate the invention rather than to limit its scope.

### Example 1

Encapsulation of ASO in Lipid Nanoparticles Targeted to the Brain with FC5 or IGF1R Single Domain Antibodies
Loading of ASO in Lipid Nanoparticles Targeted to the Brain with FC5 or IGF1R Single Domain Antibodies

Now referring to FIG. 1, which is a graphical representation of a pegylated lipid nanoparticle encapsulating an antisense oligonucleotide, conjugated to an FC5-sdAb (SEQ ID NO: 32) (Muruganandam et al, 2002) through a DSPE-PEG₂₀₀₀-DBCO moiety, according to an embodiment of the present invention. The FC5-sdAb comprises an added O-glycosylation sequon (SEQ ID NO: 33) to which an azido group is added through a conjugated O-glycan. Dlin-MC3-DMA, DSPC, cholesterol, PEG₂₀₀₀-lipid and DSPE-PEG₂₀₀₀-DBCO were solubilized in ethanol at a molar ratio of 40:10:40:9.2:0.8 respectively and a total lipid concentration of 12 mM. Molar ratio can be adjusted to 50:10:37.5:2.1:0.4 Dlin-MC3-DMA can be replaced by other cationic lipids, including ALC-0315 or SM102 or similar. Various lipid lengths can be use as anchor for the PEGs. DBCO can be replaced by other click chemistry moieties like BCN or TCO. The antisense oligonucleotides (ASO-T * mA * mA * T * A C G TAAGTGTC * A * C * mA * mA, wherein: m is a 2′-OMe base; and * is a phosphorothioate (PS) backbone; SEQ ID NO: 21) were solubilized in 25 mM acetate, pH 4 at a concentration of 0.23 mg/ml. Alternatively, mRNA (fLuc-SEQ ID NO: 20) were solubilized in 65 mM acetate, pH 4 at a concentration of 0.12 mg/ml. Using the Nanoassemblr Ignite microfluidic system (Precision Nanosystem™ Inc.), oligonucleotides/aqueous and lipids/ethanol were mixed at a ratio of 3:1 and a total flow rate of 12 ml/min. Nitrogen (N) to phosphate (P) (N/P) ratio of 3 for ASO or siRNA, can be adjusted to 6 for mRNA by changing the oligonucleotide concentration or ethanol/aqueous volume ratios. The ethanol was removed by dialysis in 1000 volumes of 0.9% (w/w) NaCl, for 24 h, using MW cut-off of 300 kDa and concentrated to 1.5 ml using Amicon™ filter MW cut-off of 100 kDa at 4000 G. A 2-fold molar excess of single domain antibody-glycan-azide (over the DSPE-PEG₂₀₀₀-DBCO) was added to the vesicles and conjugated for 24 h at 4° C. Unconjugated antibodies were removed by dialysis in 1000 volumes of PBS, pH 7.4 at 4° C. for 24 h using MW cut-off of 300 kDa. Oligonucleotide payload was determined using QuantIt™ Oligreen™.

Alternatively, antibodies can be post-inserted into pre-formed LNPs (FIG. 2). For a formulation of 14 mg of lipids (20 μmol), 10 nmol of DSPE-PEG₂k-DBCO were dried under vacuum for 1 h. The thin lipid film was hydrated with 200 μl PBS for 30 min at RT and sonicated 5 min to form micelles. Micelles were conjugated by click chemistry with 10 nmol of antibody-azide for 24 h at 4° C. The conjugated micelles were then post-inserted in 5 ml of LNPs for 45 min at 50° C. LNPs were finally cooled down for 15 min and then concentrated using Amicon 100K.

Nanoparticle diameter and concentration were determined using Dynamic and Static light scattering (Malvern) or Nanoparticles tracking analysis (Zetaview). The concentration of antibodies was determined by fluorescamine assay.

DLin-MC3-DMA was from Medkoo Biosciences™. Cholesterol, lipids, PEGs and Fluorescamine were from Sigma™. Dialysis membranes were from Spectrum Labs™. QuantIt™ Oligreen™ for ssDNA labeling was purchased at Thermosfisher™. Gold nanoparticles were from Nanoprobes™.

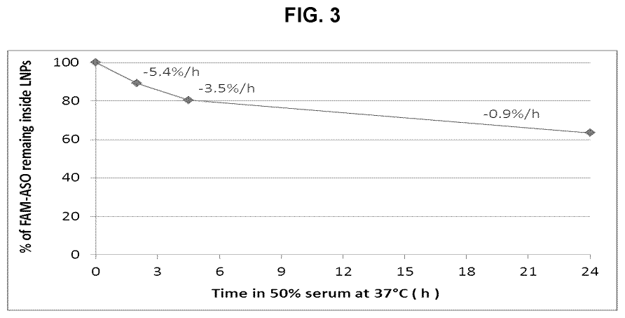

Now referring to FIG. 3. FIG. 3 illustrates LNP stability/cargo shows that LNP stability/cargo release in vitro by measuring cargo stability in 50% serum at 37° C.

### Example 2

### In Vitro Rat BBB Transmigration of FC5 SDAB-Lipid Based Nanoparticles

### Culture of Rat Brain Endothelial Cell Line

An immortalized adult rat brain microvascular endothelial cell line, SV40-immortalized adult rat brain endothelial cells (SV-ARBEC) was used for cellular internalization studies and in vitro transcytosis assays. SV-ARBEC cells were grown in M199-based feeding media (316-010-CL, Wisent, St-Bruno, Quebec) containing: 0.25% Peptone (P-5905), 0.9% d-glucose (G-8769), BME Amino Acids (B6766), BME Vitamins (B6891)—all from Sigma-Aldrich (St. Louis, MO, USA); 10% heat-inactivated fetal bovine serum (SH30396.03, Hyclone, Fisher Scientific, Ottawa, ON, USA) and antibiotic/antimycotic as previously described.

### BBB Model In Vitro

In vitro BBB permeability assays were performed using recently described protocols (Farrington et al. 2014; Webster et al. 2016). In brief, SV-ARBEC were seeded at 80 000 cells/membrane on rat tail collagen coated 0.83 cm² Falcon cell inserts, with 1 μm pore size (353103, Corning, Durham, NC, USA) in 1 mL SV-ARBEC feeding media without phenol red. The inserts were placed in the wells of a 12-well tissue culture plate containing 2 mL of 50:50 (v/v) mixture of SV-ARBEC feeding media without phenol red and rat astrocyte-conditioned media to generate a model of the BBB in vitro. Upon culturing, a barrier phenotype develops, restricting the passage of molecules between chambers; permeability was monitored, and the cultures used only when Pₑ[sucrose] was between 0.4 and 0.6 [×10⁻³] cm/min. Transport experiments were performed by adding an equimolar mixture (1.25 μM) of antibodies to the top chamber and by collecting a 100 μL aliquot from the bottom chamber at 90 min for simultaneous quantification of both the antibodies using the multiplexed SRM method.

### In Vitro Rat BBB Transmigration of IGF1R-sdAb or FC5-sdAb Lipid-Based Nanoparticles Containing ASO-Cy5

SV-ARBEC cells are seeded on 1 μm PET cell culture inserts in 12 well format in 1 mL of SV-ARBEC specific in-house growth medium. Bottom chamber is filled with 1 mL growth medium and 1 mL of rat astrocyte condition medium. Cells are grown for 6 days, and transmigration assay is performed on day 6.

The tightness of the cell monolayer is assessed using C-14-Sucrose permeability and calculating permeability coefficient (Pₑ). If the Pₑ of cells is within normal range of Pₑ<0.6×10⁻³ cm/min the assay is performed.

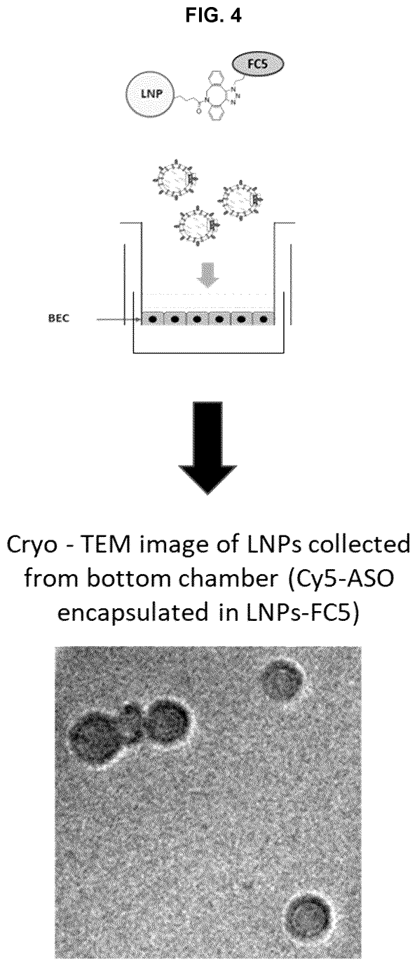

Each condition is run in at least triplicate and time points of 30 min, 60 min, 90 min, 3 hrs, and 24 hrs. 200 μL aliquots are collected from the bottom chamber straight into black 96 well plate for further analysis. FIG. 4 shows an illustration of the assay described above (left panel) and shows a cryo-transmission electron microscopy (TEM) image of LNPs-FC5 containing encapsulated ASO-Cy5, collected from a bottom chamber, according to the assay (right panel). These results support the hypothesis that FC5-LNPs stay intact as they transmigrate across the in vitro blood brain barrier.

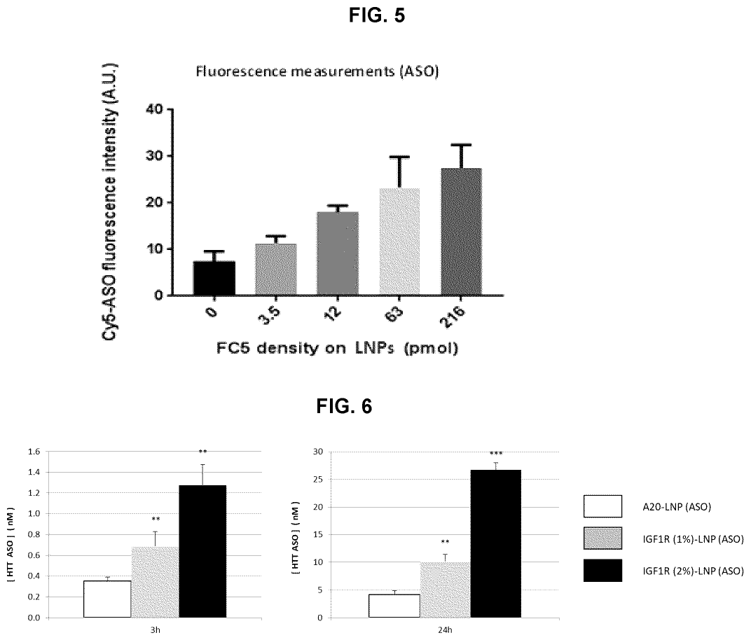

FIG. 5 illustrates the effect of increasing FC5 density on LNPs on the amount of encapsulated Cy5-ASO crossing the BBB model in vitro. Increased FC5 density results in increased fluorescence. FIG. 6 illustrates the effect of increased density of IGF1R-sdAb on LNPs on the efficiency of BBB transmigration and HTT ASO delivery in vitro. Increased IGF1R-sdAb density results in increased fluorescence. These results show that increasing the number of antibodies per nanoparticle demonstrates increased transmigration across the in vitro BBB, as measured by the fluorescence tag, Cy5, linked to the ASO cargo.

### Example 3

Detection of FC5 in LNPS-FC5 by Western Blot after Passage to the Bottom Chamber of the In Vitro Rat BBB Model

### Determination of FC5 Concentration to LNPs by Western Blot

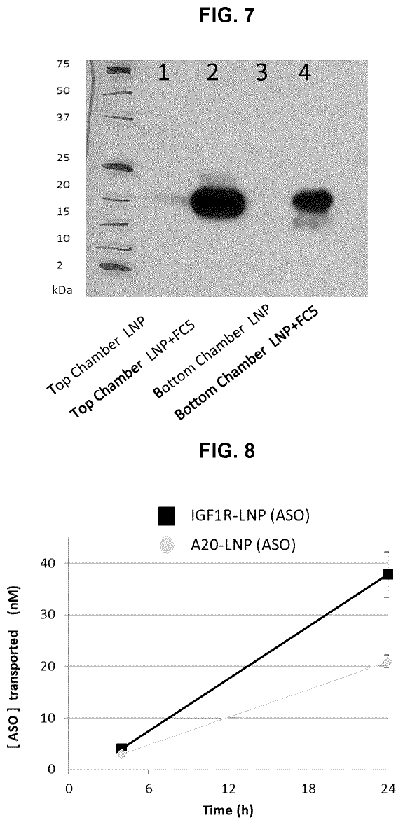

LNP-FC5 were diluted 1/200-1/2000 and 0.1-2 pmol of FC5 standard were put in 30 μl aliquots for semi-quantitative comparison. The 30 μl aliquots were incubated with 10 μl of 4× Laemli loading buffer for 30 min at 50° C. Samples were loaded in 50 μl-wells precasted 12% polyacrylamide gel (Biorad) and electrophoresed for 2 h at 100V in Tris-Glycine buffer. Bands were transferred on 0.45 μm PVDF membrane for 1 h at 1A. Membranes were blocked in TRIS buffered saline containing X % tween-20 (TBST) with 5% (w/w) non-fat milk with 0.5% (v/v) triton X-100 for 30 min at room temperature (RT) and then incubated overnight at 4° C. with 3F7 anti-FC5 diluted 1/1200 TBST. Then, primary antibody was washed 3×15 min at RT in TBST. Membrane was then incubated with Anti-mouse-HRP diluted 1/25000 in TBST with 5% (w/w) non-fat milk with 0.5% (v/v) triton X-100 for 1.5 h at RT. Then, secondary antibody was washed 3×15 min at RT in TBST. The HRP was reacted with 5 ml of ECL reagent for 1 min and exposed to a radiographic film for about 1 sec to 1 min. Now referring to FIG. 7, which shows that FC5 is detected by Western analysis in the bottom chamber. These results indicate that in this in vitro system, which simulates and acts similar to brain: blood compartments, the FC5 antibody has crossed the simulated brain endothelial cells and has transmigrated, similar to the in vivo inside the brain.

In parallel inserts (FIG. 8) containing SV-ARBEC cell, IGF1R-sdAb LNP and non-targeted LNPs containing ASO were added in separate wells. The transport of the LNPs, as measured indirectly, via ASO concentration in the bottom chamber was monitored over time up to 24 hrs. The IGF1R sdAb-LNP containing ASO demonstrated a faster rate of transport compared to the non-targeted control.

### Example 4

HTT Gene Knock-Down in Hd Patient Lymphocytes after Transport Across Rat BBB In Vitro

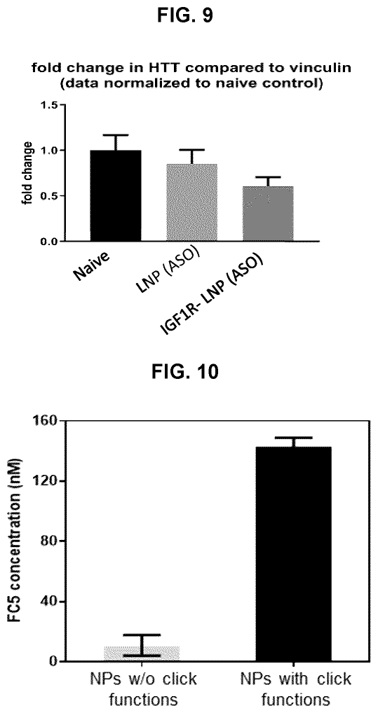

In parallel inserts containing SV-ARBEC cells, constructs of interest were placed over Huntington's (HHT) disease patient derived lymphocytes cells growing at the bottom of the wells. The purpose of this experimental design is to assess a direct effect of IGF1R-sdAb-LNP containing ASO on lymphocytes cells. Inserts are removed after 3 h and cells are harvested 48 h post transmigration assay and analyzed by western blot. FIGS. 8 and 9 shows that HTT gene knock-down in HHT disease patient lymphocytes after transport across BBB in vitro. The FIG. 8 panel show HHT ASO transport over time, while FIG. 9 shows the fold-change decrease in HHT caused by the IGF1R-sdAb-LNP containing ASO. These results show that the IGF1R-sdAb-LNPs were able to deliver the ASO across the in vitro BBB over the course of the experiment. The ASO from the LNP that had crossed the in vitro BBB were also able to knockdown Huntington protein (HTT) in patient derived lymphocytes.

### Example 5

### Determination of FC5 Concentration to LNPS by Multiple Reaction Monitoring Using Qtrap

FC5-azide (20 nmol) were added to LNPs decorated or not with DBCO (3 nmol) for 20 h at 4° C. Unconjugated FC5 (total 100 nmol) were removed by dialysis in 4 L for 48 h at 4° C. The amount of residual FC5 in LNPs ‘without click’ was compared to the amount of FC5 conjugated to LNPs ‘with click’ by Multiple reaction monitoring (MRM) using QTRAP. The results are shown in FIG. 10, which illustrates the antibody concentration/levels and visualization of attached antibody upon conjugation to LNPs.

### Example 6

### Biodistribution of IGF1R or FC5 SDAB-ASO-Nanoparticles

The in vivo biodistribution of Alexa 780 labeled FC5-ASO-Nanoparticles following a single intravenous tail vein was assessed. Administration was assessed in C57BL/6 male mice (n=4, per group). A 200 μl dose of Alexa 780 labeled FC5-ASO-Nanoparticles was injected. Animals were subjected to in vivo imaging studies using an IVIS Kinetic small animal imager (Perkin Elmer™, Waltham, Massachusetts, United States). Animals were imaged at various time points and then organs were assessed ex-vivo for fluorescence. Total or average fluorescence intensity data was determined from select regions of interest (ROI) using the Living Image 4.1 software (Perkin Elmer, Waltham, Massachusetts, United States).

### Example 7

### Assessment of In Vivo Brain Delivery of IGF1R-SDAB-ASO-LNP-CY7 by Optical Imaging

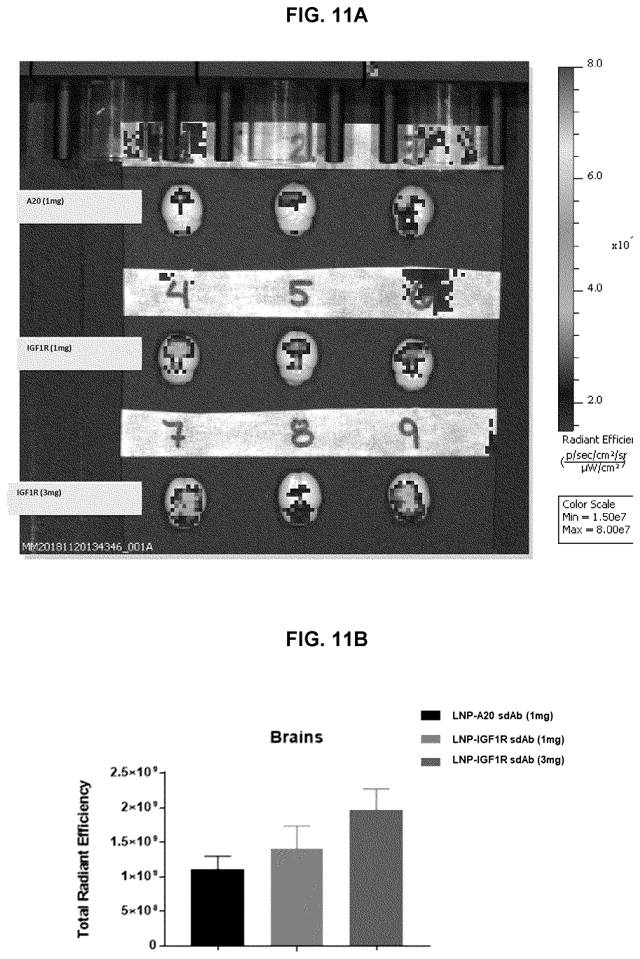

1 mg of A20 (SEQ ID NO:34) or 1 mg or 3 mg of IGF1R-sdAb-azide were conjugated to 8 mg of lipids, including 0.25 mol % of Cy7-DPPE. 2.5 mg of LNPs in a volume of 250 μl were injected intravenously in each mouse. Mice were perfused with saline at 6 h and brains were imaged ex vivo using IVIS Lumina III at Ex/Em 740 (20)/790 (40) nm. FIGS. 11A and B show the ex vivo brain delivery of A20-LNPs and IGF1R-sd-Ab-ASO-LNP-Cy7 by optical imaging. Improved brain targeting with higher density of IGF1R-sd-Ab-ASO-LNP-Cy7 is observed, in both the top and bottom panels. These results show that increasing the density of IGF1R-sdAb on nanoparticles results in increased delivery of Cy7 dye in the brain. Cy7 is linked to external surface of the LNPs.

### Example 8

### Distribution of IGF1R SDAB Brain Targeted Vs Non-Targeted LNPS (Tail Vein Injection) Using Near-Infrared Fluorescence Imaging

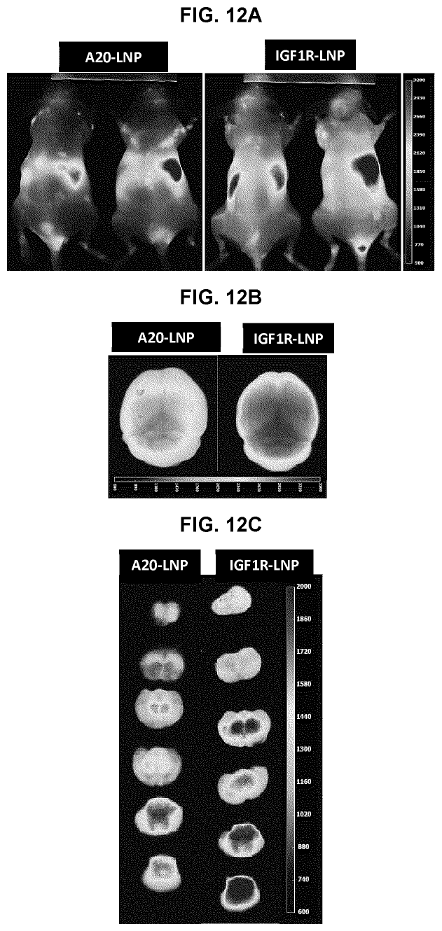

LNPs composed of DSPE-PEG₂₀₀₀-CF770 and DSPE-PEG₂₀₀₀-DBCO were conjugated with an excess IGF1R-azide or A20-azide for 24 h and then dialyzed overnight in phosphate buffered saline (PBS) using a molecular weight cut off of 300 kDa. Both LNPs were concentrated, and fluorescence normalized before intravenous injection of 250 μl. Mice were imaged after 4 h using the NIRII spectral Imager using Ex/Em 740 (20)/820 nm long pass filter (LP). Then, mice were perfused with saline and dissected brains were imaged ex vivo. Brains were also put in a mold and cut in 6×2 mm thick slices using 5 razor blades and imaged (Imaged with a NIRII spectral imager). Now referring to FIGS. 12A to C, which show the distribution of IGF1R sdAb brain targeted vs non-targeted (A20) LNPs after tail vein injection in vivo to ex vivo.

### Example 9

### Distribution of FC5 SDAB Brain Targeted Vs Non-Targeted LNPS (Tail Vein Injection) Using Near-Infrared Fluorescence Imaging

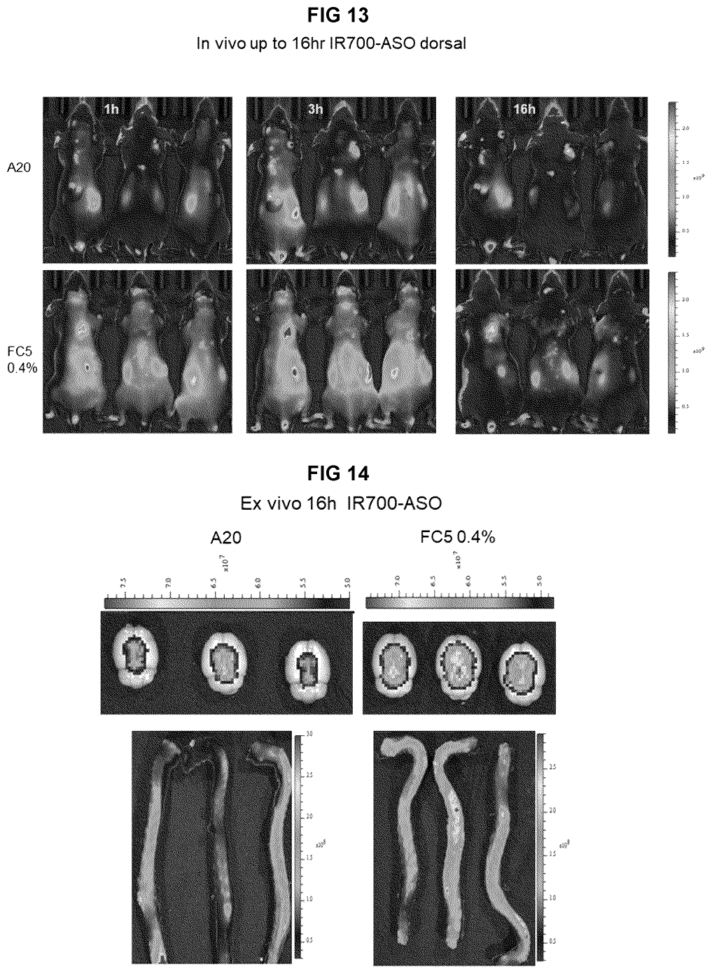

LNPs composed of DSG-PEG₂₀₀₀, ALC-0315 ionizable cationic lipid, cholesterol and DSPC containing ASO-IR700 cargo and labeled with a 815 nm lipid dye were post-inserted with DSPE-PEG-DBCO-FC5 (or A20) micelles to produce brain targeted and non-targeted LNPs, respectively. Both LNPs were concentrated, and fluorescence normalized before intravenous injection of 250 ul. Mice were imaged up to 16 hrs using Ex/Em 740/790 nm in the IVIS Lumina™ III animal imager. Then, mice were perfused with saline and dissected brains were imaged ex vivo. Now referring to FIGS. 13 and 14, which shows the in vivo (FIG. 13) biodistribution of the cargo ASO-IR700 at various time points up to 16 hrs between the FC5 and A20 conjugated LNPs after tail vein injection. In FIG. 14, the ex vivo brain and spine distribution of ASO-IR700 contained within FC5 sdAb brain targeted vs non-targeted (A20) LNPs after tail vein injection is shown.

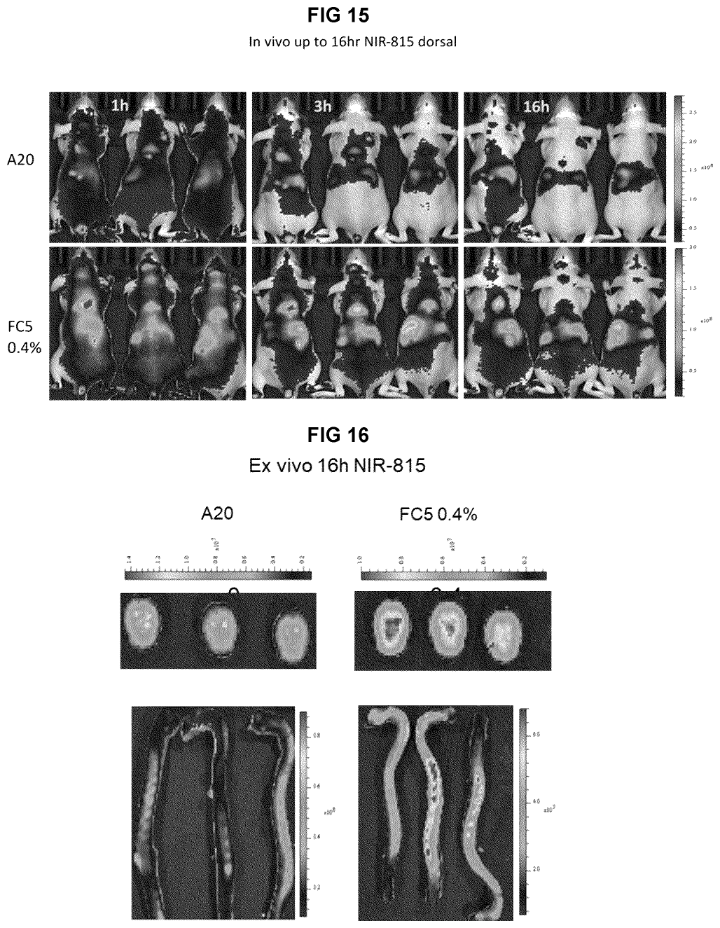

Now referring to FIGS. 15 and 16, which shows the in vivo (FIG. 15) biodistribution of the 815 fluorescently tagged LNP at various time points up to 16 hrs between the FC5 and A20 conjugated LNPs after tail vein injection. In FIG. 16, the ex vivo brain and spine distribution of FC5 sdAb brain targeted vs non-targeted (A20) LNPs after tail vein injection is shown.

### Example 10

Near-Infrared Fluorescence Imaging of Ex-Vivo Brain Slices of Mice after Intracarotid Injection of FC5-ASO-LNPS

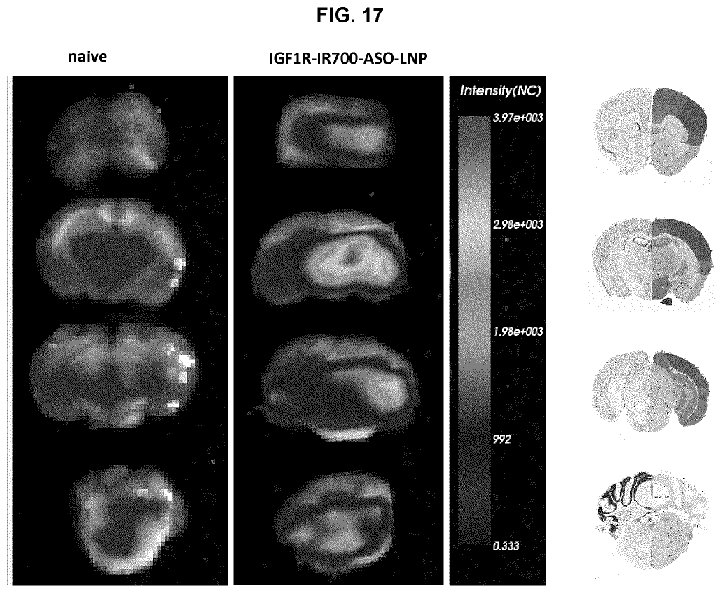

IGF1R sdAb-LNP loaded with IR700-ASO (20 nmol) were infused in mouse carotid according to the technique of Wael Alata (Alata, W., et al., 2014). After 4 h, mice were perfused with 20 ml of saline through lower left heart ventricle and dissected brains were put in a mold and sliced in 4 quarters using 3 razor blades. 3 mm thick sections were imaged with IVIS using Ex/Em 660 (20)/710 (40) nm. FIG. 17 shows the near-infrared fluorescence imaging of ex vivo brain slices of mice after intracarotid injection of IGF1R sdAb-ASO-IR700-LNPs, confirming specific localization of the LNP to the brain.

### Example 11

Bioluminescence Imaging of FC5-FLUC mRNA-LNP

ALC-0315, DSPC, cholesterol, PEG₂₀₀₀-lipid and DSPE-PEG₂₀₀₀-DBCO were solubilized in ethanol at a molar ratio of 50:10:36.3:3.3:0.4 respectively and a total lipid concentration of 10 mM. fLuc mRNA (SEQ ID NO: 20) was solubilized in 65 mM acetate, pH 4 at a concentration of 93 μg/ml. Using the Nanoassemblr Ignite microfluidic system (Precision Nanosystem™ Inc.), oligonucleotides/aqueous and lipids/ethanol were mixed at a ratio of 3:1 (Nitrogen (N) to phosphate (P) ratio of 6) and a total flow rate of 10 ml/min. The ethanol was removed by dialysis in 1000 volumes of 0.9% (w/w) NaCl, for 24 h, using MW cut-off of 300 kDa and concentrated to 1.5 ml using amicon filter MW cut-off of 300 kDa at 4000 G. A 2-fold molar excess of FC5 or A20 single domain antibody-glycan-azide (over the DSPE-PEG₂₀₀₀-DBCO) was added to the vesicles and conjugated for 24 h at 4° C. Unconjugated antibodies were removed by dialysis in 1000 volumes of PBS, pH 7.4 at 4° C. for 24 h using MW cut-off of 300 kDa.

D-Luciferin (PerkinElmer) was injected subcutaneously into mice at a dose of 300 mg/kg body weight. At time=20 min, the mice were placed on the imaging stage and photons emitted from the head were visualized using IVIS Lumina series III (Perkin Elmer™) and the total amount of bioluminescence was calculated using Living Image® software 4.7 (Perkin Elmer™).

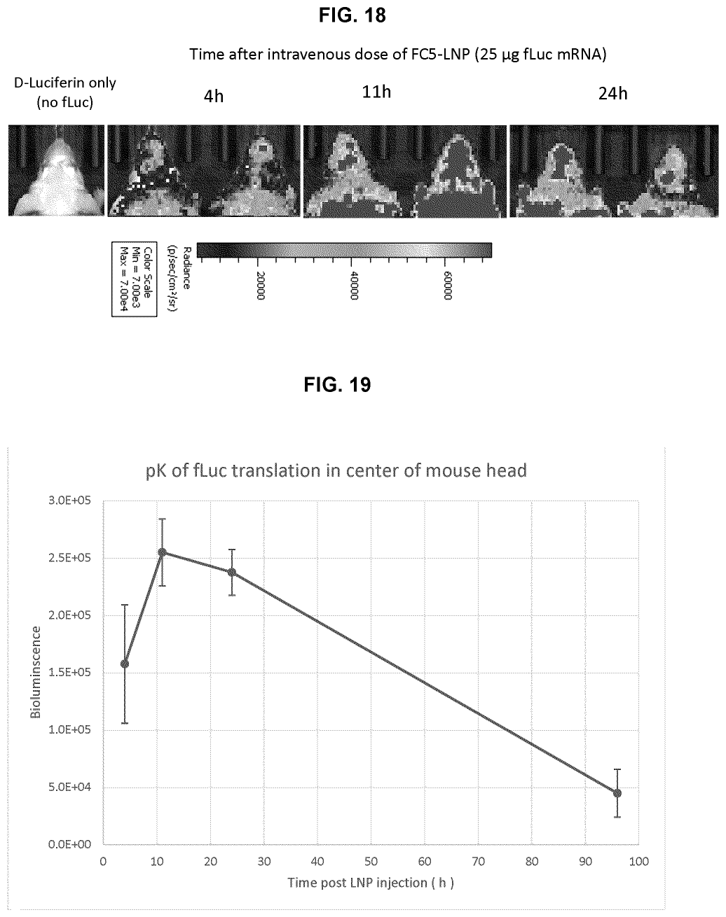

FIG. 18 illustrates the delivery and expression of fLuc mRNA (25 μg) in mouse head at 4, 12 and 24 h following intravenous injection of FC5-mRNA-LNPs, and show increase expression as time increases. Luciferase expression was visualized by injecting D-luciferin SC injection (300 mg/kg) at various time points followed by bioluminescence imaging using an IVIS Lumina Ill whole body imager (Perkin Elmer). Bioluminescence images taken at various time points (4, 11 and 24 h) post IV injection of FC5-mRNA-LNPs.

FIG. 19 shows the quantification of head kinetics of delivery and expression of fLuc mRNA at 4, 11, 24 and 96 h following intravenous injection of FC5-mRNA-LNPs. Expression/luminescence reaches a peak at about 11 h. Graph of bioluminescence intensity over time with a peak mRNA expression around 11 hrs. Expression continues high until 24 hrs, and then decreases over time up to 96 hrs.

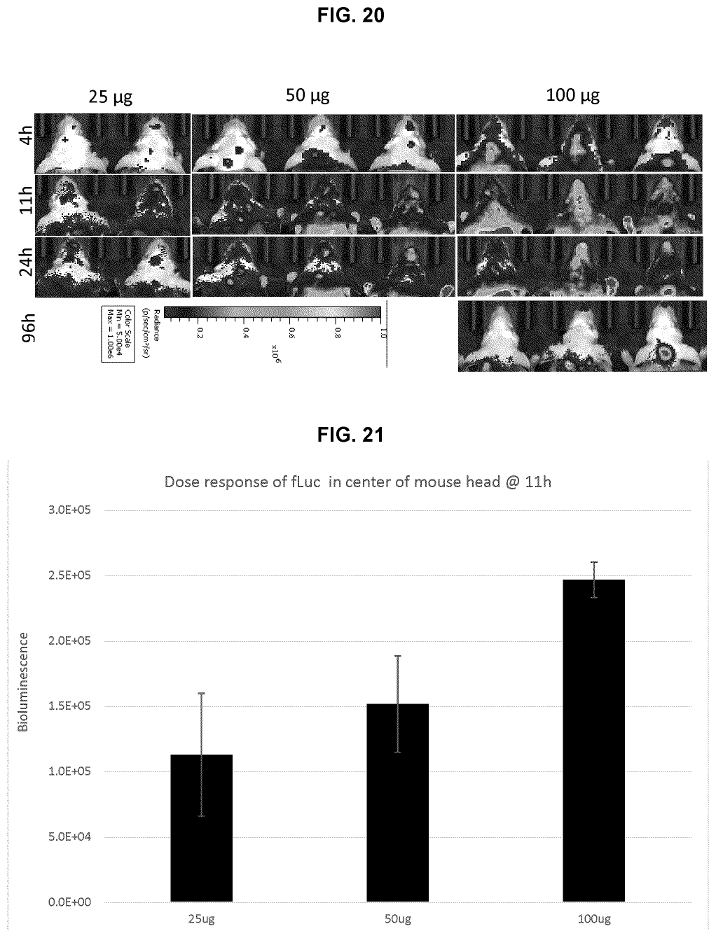

FIG. 20 illustrates the dose response of the delivery and expression of fLuc mRNA in mouse head following intravenous injection of FC5-mRNA-LNPs containing 25, 50 or 100 μg of mRNA, over a period of 96 h. Luciferase expression was visualized by injecting D-luciferin SC injection (300 mg/kg) at various time points followed by bioluminescence imaging using an IVIS Lumina III whole body imager (Perkin Elmer). The figure shows head bioluminescence images at 11 hrs post IV injection of FC5-targeted mRNA LNPs using different mRNA dosages (25, 50 and 100 μg).

FIG. 21 shows the quantification of the dose response for delivery and expression of fLuc mRNA in mouse head following intravenous injection of FC5-mRNA-LNPs containing 25, 50 or 100 μg fLuc mRNA. The graph compares the bioluminescence level in the mouse head between FC5 targeted and A20 (negative control) mRNA-LNPs at 11 hrs post IV injection (N=4 per group).

### Example 12

### Improving LNP PK Improves Brain Delivery for FC5 Targeted ASO-LNPS

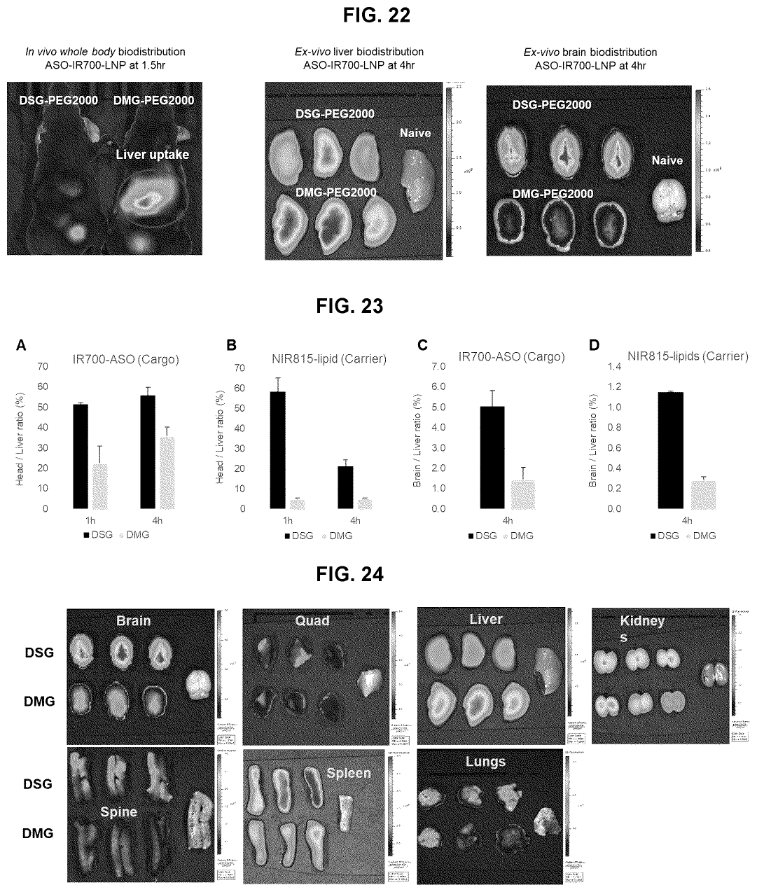

Now referring to FIG. 22. FIG. 22 (left panel) shows the in vivo optical image at 1.5 hrs post IV injection of ASO fluorescently labeled with IR700 encapsulated within FC5 targeted LNPs and containing either 2.5% DMG-PEG (left) or 2.5% DSG-PEG (right). The results show the increase fluorescence intensity in animals injected with the DSG-PEG containing LNPs vs. those injected with DMG-PEG. FIG. 22 (middle panel) and (right panel) show ex vivo optical images of ASO-IR700 fluorescence at 4 hrs after IV injection of FC5 targeted ASO-LNPs containing either 2.5% DMG-PEG or 2.5% DSG-PEG in the mouse liver or brain, respectively. The results show the increase fluorescence intensity in animals injected with the DSG-PEG containing LNPs vs. those injected with DMG-PEG.

FIGS. 23A, B shows the quantification of the NIR815 lipid tag (20A) or ASO-IR700 cargo (20B) following intravenous injection of ASO-LNPs containing either DSG-PEG₂₀₀₀ or DMG-PEG₂₀₀₀ at 4 hrs in vivo. FC5 targeted and DSG-PEG containing LNPs demonstrated increased delivery to the brain of both the ASO cargo and LNP carrier.

FIGS. 23C, D shows the quantification of the NIR815 lipid tag (20C) or ASO-IR700 cargo (20D) following intravenous injection of ASO-LNPs containing either DSG-PEG₂₀₀₀ or DMG-PEG₂₀₀₀ at 4 hrs in ex vivo brains. FC5 targeted and DSG-PEG containing LNPs demonstrated increased delivery to the brain of both the ASO cargo and LNP carrier.

FIG. 24 shows the quantification of the NIR815 lipid tag following intravenous injection of ASO-LNPs containing either DSG-PEG₂₀₀₀ or DMG-PEG₂₀₀₀ at 4 hrs in ex vivo brains, quad muscle, liver, kidney, spine, spleen and lungs. FC5 targeted and DSG-PEG containing LNPs demonstrated increased delivery to the spine of both the ASO cargo and LNP carrier.

### Example 13

Distribution of Luciferase Expression of FC5 SDAB Brain Targeted Vs Non-Targeted LNPS (Tail Vein Injection) Containing Luciferase mRNA Using Bioluminescence Imaging

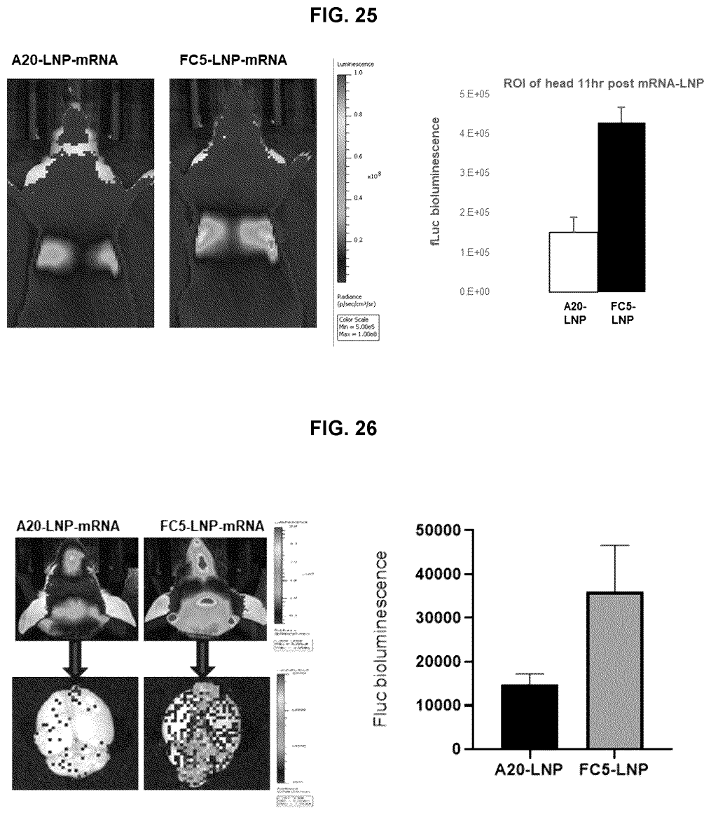

LNPs composed of DSG-PEG₂₀₀₀, ALC-0315 ionizable cationic lipid, cholesterol and DSPC containing firefly luciferase mRNA were post-inserted with DSPE-PEG-DBCO-FC5 (or A20) micelles to produce brain targeted and non-targeted LNPs, respectively. Mice were imaged at 11 hrs using an open filter setup in the IVIS Lumina™ III animal imager. Then, mice were perfused with saline and dissected brains were imaged ex vivo. Now referring to FIGS. 25 and 26, which shows the in vivo (FIG. 25, left panel) luciferase mRNA expression at 11 hrs and region of interest quantification (FIG. 25, right panel) between the FC5 and A20 conjugated LNPs after tail vein injection. In FIG. 26 (left panel), the ex vivo whole brain bioluminescence at 11 hrs post injection of FC5 sdAb brain targeted vs non-targeted (A20) LNPs is shown. In FIG. 26 (middle panel), the individual animal bioluminescence from brain homogenates at 11 hrs post injection of FC5 sdAb brain targeted vs non-targeted (A20) LNPs is shown. In FIG. 26 (right panel), a bar graph depicting the average bioluminescence signals from brain homogenates at 11 hrs post injection of FC5 sdAb brain targeted vs non-targeted (A20) LNPs is shown. FC5 targeted LNPs can deliver an increased amount mRNA to the brain.

### Example 14

Gene Editing of A19 Transgenic Mice Using Cre Recombinase mRNA Encapsulated in FC5 SDAB Brain Targeted Vs Non-Targeted LNPS (Tail Vein Injection) Using Fluorescence Imaging

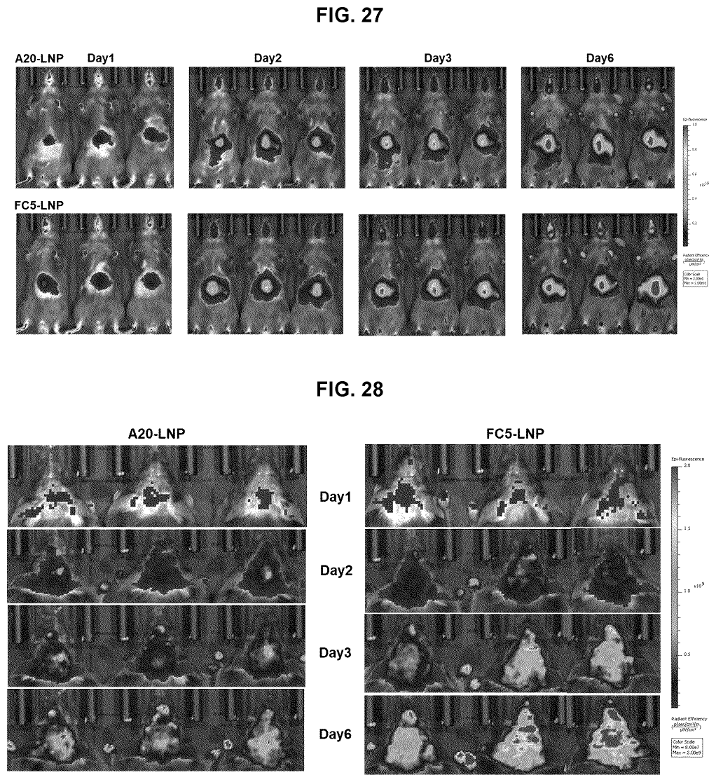

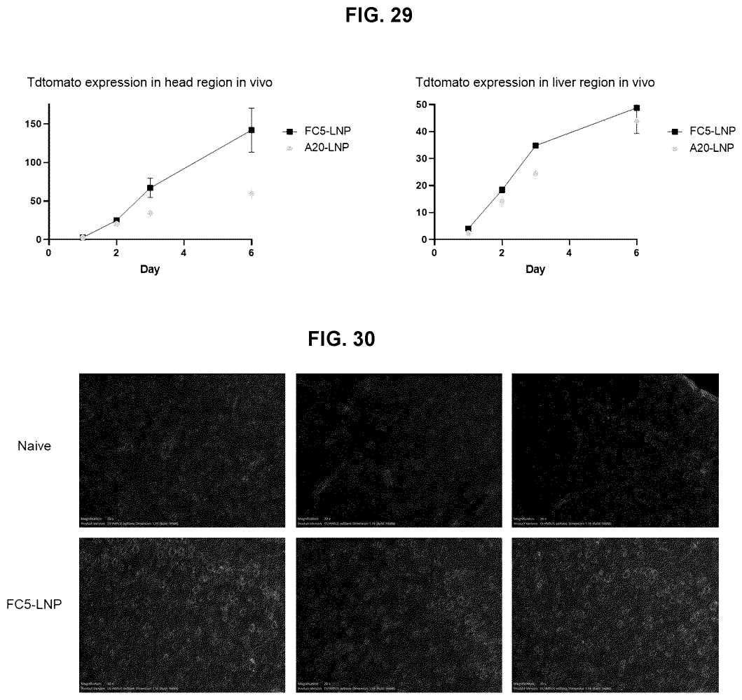

Ai9 is a Cre reporter tool strain designed to have a loxP-flanked STOP cassette preventing transcription of a CAG promoter-driven red fluorescent protein variant (tdTomato). Ai9 mice express robust tdTomato fluorescence following Cre-mediated recombination. 1500 μg of Cre mRNA (SEQ ID NO: 35) was encapsulated in 28 mg of lipids using the Nanoassemblr™ microfluidic system. LNPs were composed of DSG-PEG₂₀₀₀, ALC-0315 ionizable cationic lipid, cholesterol and DSPC containing Cre recombinase mRNA were post-inserted with DSPE-PEG-DBCO-FC5 (or A20) micelles to produce brain targeted and non-targeted LNPs. Ethanol was removed by dialysis before post-inserting targeting antibodies. 150 μg of encapsulated mRNA was injected intravenously per mouse. In vivo expression of tdTomato was assessed by imaging mice at Day 1, 2, 3 and 6, using the IVIS (PerkinElmer) with Ex 560 nm, Em 620 nm in the mouse whole mouse ventral body (FIG. 27) and in the head region (FIG. 28). FC5 targeted LNPs containing Cre recombinase mRNA demonstrated an increased signal in the head region compared to the nontargeted A20 control LNPs (FIG. 29, left panel).

At day 6, mice were perfused with heparinized saline and brains were collected and flash frozen. Brains were post fixed in 10% neutral buffered formalin and fixed for 48 hrs, then then transferred into 70% ethanol. The tissue was embedded in paraffin wax and cut into 10 μm sections on Superfrost Plus slides (Thermo Fisher). Sections were dried overnight and then subjected to immunofluorescence (IF) using a commercial anti-tdTomato goat polyclonal, 1:100, Sicgen™ ab8181-200×, followed by a FITC labeled secondary antibody (FIG. 30). FC5-LNPs containing CRE recombinase mRNA unlocked tdTomato expression in the cortex compared to the naïve Ai9 transgenic mouse brains.

While preferred embodiments have been described above and illustrated in the accompanying drawings, it will be evident to those skilled in the art that modifications may be made without departing from this disclosure. Such modifications are considered as possible variants comprised in the scope of the disclosure.

SEQUENCE TABLE

| SEQ ID NO: | Sequence | Description |
|---|---|---|
| SEQ ID NO: 1 | PTTDSTX₁PAPTTK, where X₁ is S or T | Consensus |
| | | sequon EA2 |
| SEQ ID NO: 2 | FFPX₂PGP, where X₂ is S or T | Consensus |
| | | sequon based |
| | | on Gerken et al. |
| | | (2006) |
| SEQ ID NO: 3 | GVGVX₃ETP, where X₃ is S or T | Consensus |
| | | sequon based |
| | | on sequon from |
| | | interferon alpha |
| | | 2 |
| SEQ ID NO: 4 | AAAX₄PAP, where X₄ is S or T | Consensus |
| | | sequon based |
| | | on Yoshida et |
| | | al. (1997) |
| SEQ ID NO: 5 | APALQPX₅QGAMPA, where X₅ is S or T | Consensus |
| | | sequon based |
| | | on O-linked |
| | | sequon present |
| | | in G-CSF |
| SEQ ID NO: 6 | PTTDSTTPAPTTK | Sequon EA2 |
| SEQ ID NO: 7 | PTTDSTSPAPTTK | Sequon based |
| | | on EA2 |
| SEQ ID NO: 8 | FFPTPGP | Synthetic |
| | | sequon based |
| | | on Gerken et al. |
| | | (2006) |
| SEQ ID NO: 9 | FFPSPGP | Synthetic |
| | | sequon based |
| | | on Gerken et al. |
| | | (2006) |
| SEQ ID NO: 10 | GVGVTETP | Sequon from |
| | | interferon alpha |
| | | 2 |
| SEQ ID NO: 11 | GVGVSETP | Sequon based |
| | | on sequon from |
| | | interferon alpha |
| | | 2 |
| SEQ ID NO: 12 | AAATPAP | Shortest |
| | | sequence motif |
| | | for high level |
| | | mucin-type O- |
| | | glycosylation |
| | | based on |
| | | Yoshida et al. |
| | | (1997) |
| SEQ ID NO: 13 | AAASPAP | Sequon based |
| | | on sequon from |
| | | Yoshida et al. |
| | | (1997) |
| SEQ ID NO: 14 | APALQPTQGAMPA | O-linked sequon |
| | | present in G- |
| | | CSF |
| SEQ ID NO: 15 | APALQPSQGAMPA | Sequon based |
| | | on O-linked |
| | | sequon present |
| | | in G-CSF |
| SEQ ID NO: 16 | GFKITHYTMG | CDR1 FC5 |
| SEQ ID NO: 17 | RITWGGX₁X₂TX₃YSNSVKG, where X₁ is D or K, | CDR2 FC5 |
| | X₂ is N or D, and X₃ is F, I or L | |
| SEQ ID NO: 18 | GSTSTAX₄PLRVDY, where X₄ is T or K | CDR3 FC5 |
| SEQ ID NO: 19 | X₁VQLVESGGGLVQPGGSLRLSCAASGFKITHYTMGWX₂RQA | FC5 VHH- |
| | PGKX₃X₄EX₅VSRITWGGDNTFYSNSVKGRFTISRDNSKNTX₆Y | Humanized |
| | LQMNSLRAEDTAVYYCAAGSTSTATPLRVDYWGQGTLVTVSS, | |
| | wherein X₁ = D or E, X₂ = F or V, X₃ = E or G, | |
| | X₄ = R or L, X₅ = F or W, and X₆ = L or V | |
| SEQ ID NO: 20 | ATGGAGGACGCCAAGAACATCAAGAAGGGCCCCGCCCCCT | fLUC mRNA |
| | TCTACCCCCTGGAGGACGGCACCGCCGGCGAGCAGCTGC | (Trilink |
| | ACAAGGCCATGAAGCGGTACGCCCTGGTGCCCGGCACCAT | Biotech™) |
| | CGCCTTCACCGACGCCCACATCGAGGTGGACATCACCTAC | |
| | GCCGAGTACTTCGAGATGAGCGTGCGGCTGGCCGAGGCC | |
| | ATGAAGCGGTACGGCCTGAACACCAACCACCGGATCGTGG | |
| | TGTGCAGCGAGAACAGCCTGCAGTTCTTCATGCCCGTGCT | |
| | GGGCGCCCTGTTCATCGGCGTGGCCGTGGCCCCCGCCAA | |
| | CGACATCTACAACGAGCGGGAGCTGCTGAACAGCATGGGC | |
| | ATCAGCCAGCCCACCGTGGTGTTCGTGAGCAAGAAGGGCC | |
| | TGCAGAAGATCCTGAACGTGCAGAAGAAGCTGCCCATCATC | |
| | CAGAAGATCATCATCATGGACAGCAAGACCGACTACCAGG | |
| | GCTTCCAGAGCATGTACACCTTCGTGACCAGCCACCTGCC | |
| | CCCCGGCTTCAACGAGTACGACTTCGTGCCCGAGAGCTTC | |
| | GACCGGGACAAGACCATCGCCCTGATCATGAACAGCAGCG | |
| | GCAGCACCGGCCTGCCCAAGGGCGTGGCCCTGCCCCACC | |
| | GGACCGCCTGCGTGCGGTTCAGCCACGCCCGGGACCCCA | |
| | TCTTCGGCAACCAGATCATCCCCGACACCGCCATCCTGAG | |
| | CGTGGTGCCCTTCCACCACGGCTTCGGCATGTTCACCACC | |
| | CTGGGCTACCTGATCTGCGGCTTCCGGGTGGTGCTGATGT | |
| | ACCGGTTCGAGGAGGAGCTGTTCCTGCGGAGCCTGCAGGA | |
| | CTACAAGATCCAGAGCGCCCTGCTGGTGCCCACCCTGTTC | |
| | AGCTTCTTCGCCAAGAGCACCCTGATCGACAAGTACGACCT | |
| | GAGCAACCTGCACGAGATCGCCAGCGGCGGCGCCCCCCT | |
| | GAGCAAGGAGGTGGGCGAGGCCGTGGCCAAGCGGTTCCA | |
| | CCTGCCCGGCATCCGGCAGGGCTACGGC | |
| SEQ ID NO: 21 | T * mA * mA * T * A C G T A A G T G T | ASO |
| | C * A * C * mA * mA, wherein: m is a 2′-OMe | |
| | base; and * is a phosphorothioate (PS) | |
| | backbone | |
| SEQ ID NO: 22 | QVQLVESGGGLAQAGGSLRLSCAASGRTFSMDPMAWFRQP | A20 VHH |
| | PGKEREFVAAGSSTGRTTYYADSVKGRFTISRDNAKNTVYLQ | |
| | MNSLKPEDTAVYYCAAAPYGANWYRDEYAYWGQGTQVTVSS | |
| SEQ ID NO: 23 | GGGGS | Linker sequence |
| SEQ ID NO: 24 | SSGGG | Linker sequence |
| SEQ ID NO: 25 | SSFFPTPGP | Sequon OG1: |
| | | SS linker and |
| | | sequon of SEQ |
| | | ID NO: 8 |
| SEQ ID NO: 26 | FFPTPGPFFPTPGP | Two sequons of |
| | | SEQ ID NO: 8 in |
| | | tandem |
| SEQ ID NO: 27 | SSGGGFFPTPGP | Sequon OG2: |
| | | SSGGG linker |
| | | and sequon of |
| | | SEQ ID NO: 8 |
| SEQ ID NO: 28 | SSGGGFFPTPGPFFPTPGP | Sequon OG3: |
| | | SSGGG linker |
| | | and two |
| | | sequons of SEQ |
| | | ID NO: 8 in |
| | | tandem |
| SEQ ID NO: 29 | SSGGGGVGVTETP | Sequon OG4: |
| | | SSGGG linker |
| | | and sequon |
| | | from interferon |
| | | alpha 2 |
| SEQ ID NO: 30 | GGGGAPALQPTQGAMPA | Sequon OG6: |
| | | GGGG linker |
| | | and O-linked |
| | | sequon present |
| | | in G-CSF |
| SEQ ID NO: 31 | SSGGGAAATPAP | Sequon OG5: |
| | | SSGGG linker |
| | | and shortest |
| | | sequence motif |
| | | for high level |
| | | mucin-type O- |
| | | glycosylation |
| | | based on |
| | | Yoshida et al. |
| | | (1997) |
| SEQ ID NO: 32 | DVQLQASGGGLVQAGGSLRLSCAASGFKITHYTMGWFRQ | FC5 VHH |
| | APGKEREFVSRITWGGDNTFYSNSVKGRFTISRDNAKNTV | |
| | YLQMNSLKPEDTADYYCAAGSTSTATPLRVDYWGKGTQV | |
| | TVSS | |
| SEQ ID NO: 33 | DVQLQASGGGLVQAGGSLRLSCAASGFKITHYTMGWFRQ | FC5OG2H6: |
| | APGKEREFVSRITWGGDNTFYSNSVKGRFTISRDNAKNTV | GGG linker in |
| | YLQMNSLKPEDTADYYCAAGSTSTATPLRVDYWGKGTQV | bold, sequon |
| | TVSSGGGFFPTPGPGTHHHHHHGT | underlined |
| SEQ ID NO: 34 | QVQLVESGGGLAQAGGSLRLSCAASGRTFSMDPMAWFR | A20.1-OG2: |
| | QPPGKEREFVAAGSSTGRTTYYADSVKGRFTISRDNAKNT | GGG Linker in |
| | VYLQMNSLKPEDTAVYYCAAAPYGANWYRDEYAYWGQGT | bold, sequon |
| | QVTVSSGGGFFPTPGPGTHHHHHHGT | underlined |
| SEQ ID NO: 35 | ATGGGCACCCCCAAGAAGAAGCGGAAGGTGCGGACCG | CRE mRNA |
| | ACCTGAACAGCAACCTGCTGACCGTGCACCAGAACCTG | (Trilink |
| | CCCGCCCTGCCCGTGGACGCCACCAGCGACGAGGTGC | Biotech™) |
| | GGAAGAACCTGATGGACATGTTCCGGGACCGGCAGGCC | |
| | TTCAGCGAGCACACCTGGAAGATGCTGCTGAGCGTGTG | |
| | CCGGAGCTGGGCCGCCTGGTGCAAGCTGAACAACCGG | |
| | AAGTGGTTCCCCGCCGAGCCCGAGGACGTGCGGGACTA | |
| | CCTGCTGTACCTGCAGGCCCGGGGCCTGGCCGTGAAGA | |
| | CCATCCAGCAGCACCTGGGCCAGCTGAACATGCTGCAC | |
| | CGGCGGAGCGGCCTGCCCCGGCCCAGCGACAGCAACG | |
| | CCGTGAGCCTGGTGATGCGGCGGATCCGGAAGGAGAA | |
| | CGTGGACGCCGGCGAGCGGGCCAAGCAGGCCCTGGCC | |
| | TTCGAGCGGACCGACTTCGACCAGGTGCGGAGCCTGAT | |
| | GGAGAACAGCGACCGGTGCCAGGACATCCGGAACCTG | |
| | GCCTTCCTGGGCATCGCCTACAACACCCTGCTGCGGAT | |
| | CGCCGAGATCGCCCGGATCCGGGTGAAGGACATCAGCC | |
| | GGACCGACGGCGGCCGGATGCTGATCCACATCGGCCG | |
| | GACCAAGACCCTGGTGAGCACCGCCGGCGTGGAGAAG | |
| | GCCCTGAGCCTGGGCGTGACCAAGCTGGTGGAGCGGT | |
| | GGATCAGCGTGAGCGGCGTGGCCGACGACCCCAACAA | |
| | CTACCTGTTCTGCCGGGTGCGGAAGAACGGCGTGGCCG | |
| | CCCCCAGCGCCACCAGCCAGCTGAGCACCCGGGCCCT | |
| | GGAGGGCATCTTCGAGGCCACCCACCGGCTGATCTACG | |
| | GCGCCAAGGACGACAGCGGCCAGCGGTACCTGGCCTG | |
| | GAGCGGCCACAGCGCCCGGGTGGGCGCCGCCCGGGA | |
| | CATGGCCCGGGCCGGCGTGAGCATCCCCGAGATCATGC | |
| | AGGCCGGCGGCTGGACCAACGTGAACATCGTGATGAAC | |
| | TACATCCGGAACCTGGACAGCGAGACCGGCGCCATGGT | |
| | GCGGCTGCTGGAGGACGGCGACTGA | |

### REFERENCES

Alata W, Paris-Robidas S, Emond V, Bourasset F, Calon F. Brain uptake of a fluorescent vector targeting the transferrin receptor: a novel application of in situ brain perfusion. Mol Pharm. 2014 Jan. 6; 11 (1): 243-53. doi: 10.1021/mp400421a. Epub 2013 Nov. 22. PMID: 24215184.
Arbabi-Ghahroudi, M., Desmyter, A., Wyns, L., Hamers, R., Muyldermans, S. (1997) FEBS Lett 414:521-6.
Bell, A., Wang, Z. J., Arbabi-Ghahroudi, M., Chang, T. A., Durocher, Y., Trojahn, U., Baardsnes, J., Jaramillo, M. L., Li, S., Baral, T. N., O'Connor-McCourt, M., Mackenzie, R., Zhang, J. (2010) Cancer Lett 289:81-90.
Chothia, C., Lesk, A. M. (1987) J Mol Biol 196:901-17.
Davies, J., Riechmann, L. (1996) Immunotechnology 2:169-79.
de Kruif, J., Logtenberg, T. (1996) J Biol Chem 271:7630-4.
Dumoulin, M., Conrath, K., Van Meirhaeghe, A., Meersman, F., Heremans, K., Frenken, L. G., Muyldermans, S., Wyns, L., Matagne, A. (2002) Protein Sci 11:500-15.
Eisenberg, D., Schwarz, E., Komaromy, M., and Wall, R. (1984). Analysis of membrane and surface protein sequences with the hydrophobic moment plot. J. Mol. Biol. 179:125-42.
Gerken T A, Raman J, Fritz T A & Jamison O (2006) Identification of common and unique peptide substrate preferences for the UDP-GalNAc:polypeptide alpha-N-acetylgalactosaminyltransferases T1 and T2 derived from oriented random peptide substrates. J Biol Chem 281:32403-32416.
Gottesman, M. M., and Pastan, I. (1993). Biochemistry of Multidrug Resistance Mediated by the Multidrug Transporter. Annu. Rev. Biochem. 62:385-427.
Hamers-Casterman, C., Atarhouch, T., Muyldermans, S., Robinson, G., Hamers, C., Songa, E. B., Bendahman, N., Hamers, R. (1993) Nature 363:446-8.
Hussack, G., Arbabi-Ghahroudi, M., van Faassen, H., Songer, J. G., Ng, K. K., Mackenzie, R., Tanha, J. (2011a) J Biol Chem 286:8961-76.
Hussack, G., Hirama, T., Ding, W., Mackenzie, R., Tanha, J. (2011b) PLOS One 6: e28218.
Jespers, L., Schon, O., James, L. C., Veprintsev, D., Winter G. (2004) J Mol Biol 337:893-903.
Kabat, E. A., Wu, T. T., (1991) J Immunol 147:1709-1719.
Kim, D. Y., Kandalaft, H., Ding, W., Ryan, S., van Faassen, H., Hirama, T., Foote, S., Mackenzie, R., Tanha, J. (2012) Protein Eng Des Sel 25:581-9.
Li, S., Zheng, W., Kuolee, R., Hirama, T., Henry, M., Makvandi-Nejad, S., Fjällman, T., Chen, W., Zhang, J. (2009) Mol Immunol 46:1718-26.
Merritt, E. A., Hol, W. G. (1995) Curr Opin Struct Biol 5:165-71.
Muruganandam, A., Tanha, J., Narang, S., Stanimirovic, D. (2002) FASEB J 16:240-2.
Nicaise, M., Valerio-Lepiniec, M., Minard, P., Desmadril, M. (2004) Protein Sci 13:1882-91.
Nielsen, U. B., Adams, G. P., Weiner, L. M., Marks, J. D. (2000) Cancer Res 60:6434-40.
Nuttall, S. D., Krishnan, U. V., Doughty, L., Pearson, K., Ryan, M. T., Hoogenraad, N.J., Hattarki, M., Carmichael, J. A., Irving, R. A., Hudson, P. J. (2003) Eur J Biochem 270:3543-54.
Pardridge, W. M. (1995). Transport of small molecules through the blood-brain barrier: biology and methodology. Adv. Drug Deliv. Rev. 15:5-36.
Ridgway, J. B., Presta, L. G., Carter, P. (1996) Protein Eng 9:617-21.
Samuels, B. L., Mick, R., Vogelzang, N.J., Williams, S. F., Schilsky, R. L., Safa, A. R., et al. (1993). Modulation of vinblastine resistance with cyclosporine: A phase I study. Clin. Pharmacol. Ther. 54:421-429.
Spiess, C, Zhai, Q., Carter, P. J. (2015) Mol Immunol 67:95-106.
Stanimirovic D, Kemmerich K, Haqqani A S, Farrington G K. Engineering and pharmacology of blood-brain barrier-permeable bispecific antibodies. Adv Pharmacol. 2014; 71:301-35. doi: 10.1016/bs.apha.2014.06.005. Epub 2014 Aug. 23. PMID: 25307221.
To, R., Hirama, T., Arbabi-Ghahroudi, M., Mackenzie, R., Wang, P., Xu, P., Ni, F., Tanha, J. (2005) J Biol Chem 280:41395-403.
Watanabe, T., Tsuge, H., Oh-Hara, T., Naito, M., Tsuruo, T., Gigante, M., et al. (1995). Comparative study on reversal efficacy of SDZ PSC 833, cyclosporin A and verapamil on multidrug resistance in vitro and in vivo. Acta Oncol. 34:235-41.
Webster C I, Caram-Salas N, Haqqani A S, Thom G, Brown L, Rennie K, Yogi A, Costain W, Brunette E, Stanimirovic D B. Brain penetration, target engagement, and disposition of the blood-brain barrier-crossing bispecific antibody antagonist of metabotropic glutamate receptor type 1. FASEB J. 2016 May; 30 (5): 1927-40. doi: 10.1096/fj.201500078. Epub 2016 Feb. 2. PMID: 26839377.
Yoshida A, Suzuki M, Ikenaga H & Takeuchi M (1997) Discovery of the shortest sequence motif for high level mucin-type O-glycosylation. J Biol Chem 272:16884-16888.
Zhang, J., Li, Q., Nguyen, T. D., Tremblay, T. L., Stone, E., To, R., Kelly, J., Mackenzie C. R. (2004a) J Mol Bio/341:161-9.
Zhang, J., Tanha, J., Hirama, T., Khieu, N. H., To, R., Tong-Sevinc, H., Stone, E., Brisson, J. R., Mackenzie, C. R. (2004b) J Mol Biol 335:49-56.
Zhu, X., Wang, L., Liu, R., Flutter, B., Li, S., Ding, J., Tao, H., Liu, C., Sun, M., Gao, B. (2010) Immunol Cell Biol 88:667-75.

1. A pharmaceutical composition comprising:
a) a lipid nanoparticle operable to encapsulate a therapeutic agent, comprising a core and an external surface, said therapeutic agent being encapsulated within said core; said lipid nanoparticle having
a size of said lipid nanoparticle of from about 30 to about 80 nm, or
a pegylated lipid comprising a distearoyl-rac-glycerol (DSG)-PEG, 1,2-distearoyl-sn-glycero-3-phosphoethanolamine-N-(DSPE)-PEG-DBCO, or a combination thereof; or
a combination of: a size of from about 30 to about 80 nm and a pegylated lipid comprising a DSG-PEG, DSPE-PEG-DBCO, or a combination thereof; and
b) an antibody or antigen-binding fragment thereof operable to transmigrate the blood-brain barrier (BBB), wherein the antibody or antigen-binding fragment thereof comprises complementarity determining regions (CDR1, CDR2 and CDR3), operably linked to said external surface of said lipid nanoparticle.

2. The pharmaceutical composition of claim 1, wherein said size of said lipid nanoparticle is from about 40 to about 80 nm.

3. The pharmaceutical composition of claim 1 or 2, wherein said lipid nanoparticle comprises an ionizable cationic lipid, a helper phospholipid, cholesterol, PEG-lipid and combinations thereof.

4. The pharmaceutical composition of any one of claims 1-3, wherein said pegylated lipid further comprises a 1,2-dimyristoyl-rac-glycero-3-methoxy (DMG)-PEG, 1,2-dipalmitoyl-sn-glycero-3-phosphoethanolamine-N(DPPE)-PEG, or a combination thereof.

5. The pharmaceutical composition of any one of claims 1-4, wherein said pegylated lipid comprises a PEG group having a molecular weight of about 500 to about 5000 g/mol.

6. The pharmaceutical composition of claim 5, wherein said PEG group has a molecular weight of about 2000 g/mol.

7. The pharmaceutical composition of any one of claims 1-6, wherein said DSG-PEG is DSG-PEG₂₀₀₀.

8. The pharmaceutical composition of any one of claims 1-6, wherein said DSPE-PEG is DSPE-PEG₂₀₀₀.

9. The pharmaceutical composition of claim 4, wherein said DMG-PEG or said DPPE-PEG is DMG-PEG₂₀₀₀ and DPPE-PEG₂₀₀₀, respectively.

10. The pharmaceutical composition of claim 3, wherein said ionizable lipid is (6Z,9Z,28Z,31Z)-Heptatriaconta-6,9,28,31-tetraen-19-yl 4-(dimethylamino) butanoate (DLin-MC3-DMA), 2-[2,2-bis[(9Z,12Z)-octadeca-9,12-dienyl]-1,3-dioxolan-4-yl]-N,N-dimethylethanamine (DLin-KC2-DMA), [(4-hydroxybutyl)azanediyl]di(hexane-6,1-diyl)bis(2-hexyldecanoate) (ALC-0315), Heptadecan-9-yl 8-{(2-hydroxyethyl)[6-oxo-6-(undecyloxy)hexyl]amino}octanoate (SM-102), and combinations thereof.

11. The pharmaceutical composition of claim 3, wherein said helper phospholipid is 1,2-distearoyl-sn-glycero-3-phosphocholine (DSPC), 1,2-dioleoyl-sn-glycero-3-phosphocholine (DOPC), 1,2-Dioleoyl-sn-glycero-3-phosphoethanolamine (DOPE), 1-stearoyl-2-oleoyl-sn-glycero-3-phosphocholine (SOPC) and combinations thereof.

12. The pharmaceutical composition of any one of claims 1-11, wherein said antibody or antigen-binding fragment thereof operable to transmigrate the BBB further comprises an added O-glycosylation sequon glycosylated with an O-glycan having the general formula (I): 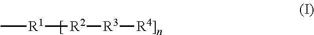
wherein
R¹ is an initial N-acetylgalactosamine (GalNAc);
n=1, or 2;
R² are each independently absent, galactose (Gal), GalNAc, N-Acetylglucosamine (GlcNAc), or a sialic acid;
R³ are each independently absent, Gal or a sialic acid; and
R⁴ are each independently absent or a sialic acid;
wherein said O-glycan is operably linked to said external surface of said nanoparticle.

13. The pharmaceutical composition of claim 12, wherein said initial GalNAc, and/or any one of said R¹, R², R³ and R⁴ is further modified with one or more chemical group.

14. The pharmaceutical composition of claim 13, wherein said chemical group is one or more of a methyl group, an acetyl group, a sulfate group, or a combination thereof.

15. The pharmaceutical composition of claim 12, wherein said sialic acid is N-Acetylneuraminic acid (Neu5Ac), 9-azido-N-Acetylneuraminic acid (9N3-Neu5Ac), N-azidoacetylneuraminic acid (Neu5NAz), or a combination thereof.

16. The pharmaceutical composition of any one of claims 12-15, wherein n=1 and R² is Gal.

17. The pharmaceutical composition of claim 16, wherein R³ is a sialic acid selected from the group consisting of Neu5Ac, Neu5NAz and 9N3-Neu5Ac, and R⁴ is absent.

18. The pharmaceutical composition of claim 12, wherein the O-glycan has the general formula (II): 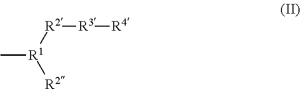
wherein
R^{2′} is Gal, or GlcNAc;
R^{3′} is Gal or a sialic acid;
R^{4′} is absent, or a sialic acid; and
R^{2″} is GlcNAc or a sialic acid.

19. The pharmaceutical composition of claim 18, wherein
a R^{2′} is Gal, a R^{3′} is a sialic acid consisting of Neu5Ac, and a R^{4′} is absent or a sialic acid consisting of 9N3-Neu5Ac; and
a R^{2″} is a sialic acid consisting of Neu5Ac.

20. The pharmaceutical composition of any one of claims 12 to 19, wherein said added O-glycosylation sequon comprise an amino acid sequence comprising:
PTTDSTX₁PAPTTK, where X₁ is S or T (SEQ ID NO: 1);
FFPX₂PGP, where X₂ is S or T (SEQ ID NO: 2);
GVGVX₃ETP, where X₃ is S or T (SEQ ID NO: 3);
AAAX₄PAP, where X₄ is S or T (SEQ ID NO: 4); and
APALQPX₅QGAMPA, where X₅ is S or T (SEQ ID NO: 5), or
combinations thereof.

21. The pharmaceutical composition of claim 9, wherein said added O-glycosylation sequon comprise an amino acid sequence comprising:
(SEQ ID NO: 6)
PTTDSTTPAPTTK,
(SEQ ID NO: 7)
PTTDSTSPAPTTK,
(SEQ ID NO: 8)
FFPTPGP;
(SEQ ID NO: 9)
FFPSPGP,
(SEQ ID NO: 10)
GVGVTETP,
(SEQ ID NO: 11)
GVGVSETP,
(SEQ ID NO: 12)
AAATPAP,
(SEQ ID NO: 13)
AAASPAP;
(SEQ ID NO: 14)
APALQPTQGAMPA,
and
(SEQ ID NO: 15)
APALQPSQGAMPA.

22. The pharmaceutical composition of any one of claims 12 to 21, wherein said added O-glycosylation sequon is at a C-terminus of said antibody or antigen-binding fragment thereof.

23. The pharmaceutical composition of any one of claims 1 to 11, wherein said antibody or antigen-binding fragment thereof operable to transmigrate the BBB, comprises
a cysteine amino acid operable to make a thioether covalent bond, and/or
an epsilon amino group operable to make an amide covalent bond,
for conjugation with said nanoparticle.

24. The pharmaceutical composition of any one of claims 1 to 11, wherein said antibody or antigen-binding fragment thereof operable to transmigrate the BBB comprises a reactive functional group for conjugation with said lipid nanoparticle.

25. The pharmaceutical composition of claim 24, wherein said reactive functional group is an azido group.

26. The pharmaceutical composition of any one of claims 12 to 25, wherein the antigen-binding fragment is a single-domain antibody (sdAb), a fragment antigen-binding (Fab), a single-chain variable fragment (scFv), or a single-chain fragment antigen-binding (scFab).

27. The pharmaceutical composition of any one of claims 12 to 26, wherein the antibody is an IgA, an IgD, an IgE, an IgG, or an IgM.

28. The pharmaceutical composition of any one of claims 12 to 27, wherein the antibody or antigen-binding fragment thereof is humanized or partially humanized.

29. The pharmaceutical composition of any one of claims 12 to 28, wherein the antibody or antigen-binding fragment thereof comprises complementarity determining regions (CDR1, CDR2 and CDR3) having the sequences:
a CDR1 sequence GFKITHYTMG (SEQ ID NO:16); CDR2 sequence RITWGGX₁X₂TX₃YSNSVKG, where X₁ is D or K, X₂ is N or D, and X₃ is F, I or L (SEQ ID NO: 17); and CDR3 sequence GSTSTAX₄PLRVDY, where X₄ is T or K (SEQ ID NO:18).

30. The pharmaceutical composition of any one of claims 12 to 29, wherein the antibody or antigen-binding fragment thereof comprises an amino acid sequence selected from the group consisting of:
(SEQ ID NO: 19)
X₁VQLVESGGGLVQPGGSLRLSCAASGFKITHYTMGWX₂RQAPG
KX₃X₄EX₅VSRITWGGDNTFYSNSVKGRFTISRDNSKNTX₆YLQM
NSLRAEDTAVYYCAAGSTSTATPLRVDYWGQGTLVTVSS,
wherein
X₁ = D or E,
X₂ = F or V,
X₃ = E or G,
X₄ = R or L,
X₅ = F or W,
and
X₆ = L or V.

31. The pharmaceutical composition of claims any one of claims 1-30, wherein said external surface comprises a functionalized cyclooctyne operably linking said antibody or antigen-binding fragment or said O-glycan of said antibody or antigen-binding fragment to said external surface.

32. The pharmaceutical composition of claim 31, wherein said O-glycan comprises a 9N3-Neu5Ac moiety operably linked to said functionalized cyclooctyne.

33. The pharmaceutical composition of any one of claims 31-32, wherein said functionalized cyclooctyne is dibenzocyclooctyne (DBCO), bicyclononyne (BCN), cyclooctyne (COT), monofluorinated cyclooctyne (MOFO), difluorocyclooctyne (DIFO), dimethoxyazacyclooctyne (DIMAC), dibenzoazacyclooctyne (DIBAC), biarylazacyclooctynone (BARAC), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), or combinations thereof.

34. The pharmaceutical composition of any one of claims 31-33, wherein said functionalized cyclooctyne is conjugated to said pegylated lipid.

35. The pharmaceutical composition of claim 34, wherein said pegylated lipid is a pegylated phospholipid.

36. The pharmaceutical composition of claim 35, wherein said pegylated phospholipid is DSPE-PEG₂₀₀₀-X¹, wherein X¹ is said functionalized cyclooctyne.

37. The pharmaceutical composition of claim 35, wherein said functionalized cyclooctyne is DBCO, BCN, COT, or combinations thereof.

38. The pharmaceutical composition of any one of claims 1-37, wherein said lipid nanoparticle comprises a molar ratio of from about 10% to about 60% of an ionizable lipid.

39. The pharmaceutical composition of claim 38, wherein said lipid nanoparticle comprises a molar ratio of from about 30% to about 50% of an ionizable lipid.

40. The pharmaceutical composition of any one of claims 1-37, wherein said lipid nanoparticle comprises a molar ratio of from about 10% to about 30% of 1,2-distearoyl-sn-glycero-3-phosphocholine (DSPC), 1,2-dioleoyl-sn-glycero-3-phosphocholine (DOPC), 1,2-Dioleoyl-sn-glycero-3-phosphoethanolamine (DOPE), 1-stearoyl-2-oleoyl-sn-glycero-3-phosphocholine (SOPC) and combinations thereof.

41. The pharmaceutical composition of claim 40, wherein said lipid nanoparticle comprises a molar ratio of from about 5% to about 10% of said DSPC, DOPC, DOPE, SOPE, or combinations thereof.

42. The pharmaceutical composition of any one of claims 1-41, wherein said lipid nanoparticle comprises a molar ratio of from about 20% to about 50% of cholesterol.

43. The pharmaceutical composition of claim 42, wherein said lipid nanoparticle comprises a molar ratio of from about 35% to 40% of cholesterol.

44. The pharmaceutical composition of any one of claims 1-43, wherein said lipid nanoparticle comprises a molar ratio of from about 1% to about 10% of distearoyl-rac-glycerol-[PEG-2000] (DSG-PEG₂₀₀₀), 1,2-distearoyl-sn-glycero-3-phosphoethanolamine-N-[PEG-2000] (DSPE-PEG₂₀₀₀), 1,2-dipalmitoyl-sn-glycero-3-phosphoethanolamine-N-[PEG-2000] (DPPE-PEG₂₀₀₀), or 1,2-dimyristoyl-rac-glycero-3-methoxy-[PEG-2000] (DMG-PEG₂₀₀₀).

45. The pharmaceutical composition of claim 44, wherein said lipid nanoparticle comprises a molar ratio of from about 1% to about 5% of distearoyl-rac-glycerol-[PEG-2000] (DSG-PEG₂₀₀₀), 1,2-distearoyl-sn-glycero-3-phosphoethanolamine-N-[PEG-2000] (DSPE-PEG₂₀₀₀), 1,2-dipalmitoyl-sn-glycero-3-phosphoethanolamine-N-[PEG-2000] (DPPE-PEG₂₀₀₀), or 1,2-dimyristoyl-rac-glycero-3-methoxy-[PEG-2000] (DMG-PEG₂₀₀₀).

46. The pharmaceutical composition of any one of claims 1-45, wherein said lipid nanoparticle comprises a molar ratio of from about 0.05% to about 2% of DPPE-PEG₂₀₀₀-DBCO, DSG-PEG₂₀₀₀-DBCO, DMG-PEG₂₀₀₀-DBCO, DSPE-PEG₂₀₀₀-DBCO, or combinations thereof.

47. The pharmaceutical composition of claim 44, wherein said lipid nanoparticle comprises a molar ratio of from about 0.05% to about 1% of DSPE-PEG₂₀₀₀-DBCO, DSG-PEG₂₀₀₀-DBCO, DMG-PEG₂₀₀₀-DBCO, DPPE-PEG₂₀₀₀-DBCO, or combinations thereof.

48. The pharmaceutical composition of any one of claims 38-47, wherein said lipid nanoparticle comprises a molar ratio of:
from about 40% to about 50% DLin-MC3-DMA, ALC-0315, or a combination thereof;
from about 5% to about 10% DSPC;
from about 35% to about 40% cholesterol;
from about 1% to about 5% DSG-PEG₂₀₀₀, DSPE-PEG₂₀₀₀, DMG-PEG₂₀₀₀, or DPPE-PEG₂₀₀₀, or combinations thereof; and
from about 0.05% to about 1% of DSPE-PEG₂₀₀₀-DBCO.

49. The pharmaceutical composition of any one of claims 1 to 48, wherein said therapeutic agent is a nucleic acid, peptide, a polypeptide, a protein, an enzyme, an antibody, an antibody fragment, or combinations thereof.

50. The pharmaceutical composition of claim 49, wherein said nucleic acid is an antisense oligonucleotide (ASO), a single stranded RNA molecule, a duplex RNA, a ministering DNA (msDNA), a DNA plasmid, or combinations thereof.

51. The pharmaceutical composition of claim 50, wherein said duplex RNA is a small interfering RNA (siRNA), a microRNA (miRNA), or a combination thereof.

52. The pharmaceutical composition of claim 50, wherein said single stranded RNA molecule is a mRNA, short hairpin RNA (shRNA), and anti-miRNA, or combinations thereof.

53. A composition comprising the pharmaceutical composition of any one of claims 1 to 52, and a pharmaceutically acceptable diluent, carrier or excipient.

54. A method of delivering a therapeutic agent across the BBB, comprising administering the pharmaceutical composition according to any one of claims 1 to 53 or a composition according to claim 55 to a subject in need thereof.

55. The method of claim 54, wherein said therapeutic agent is a pharmaceutical composition according to claim 53, and said method is for the treatment of a brain disease via gene silencing, addition or editing.

56. Use of a pharmaceutical composition according to any one of claims 1 to 52 or a composition according to claim 53, for the delivery of a therapeutic agent in the brain of a subject in need thereof.

57. The use of claim 56, wherein said therapeutic agent is a pharmaceutical composition according to claim 54, and said use is for the treatment of a brain disease via gene silencing, addition or editing.
